(19) United States
(12) Patent Application Publication
(10) Pub. No.: US 2026/0102764 A1
(43) Pub. Date: Apr. 16, 2026

(54) CHROMIUM AND ARSENIC SEPARATIONS USING POROUS ORGANIC FRAMEWORKS

(71) Applicant: The Regents of the University of California, Oakland, CA (US)

(72) Inventors: Jeffrey R. Long, Lafayette, CA (US); Adam Andrew Uliana, Brooklyn, NY (US)

(21) Appl. No.: 19/115,189

(22) PCT Filed: Oct. 4, 2023
(86) PCT No.: PCT/US2023/034428

§ 371 Date: Mar. 25, 2025

Related U.S. Application Data
(60) Provisional application No. 63/413,171, filed on Oct. 4, 2022.

(51) Int. Cl. B01J 41/12; B01J 41/04; C02F 1/42; C02F 101/10; C02F 101/22; C07C 215/68

(52) U.S. Cl. CPC B01J 41/12; B01J 41/04; C02F 1/42; C07C 215/68; C02F 2001/422; C02F 2101/103; C02F 2101/22

(57) ABSTRACT

The disclosure provides for porous organic frameworks functionalized to comprise pendant amino-dial groups and/or amino-polyol groups, and uses thereof, including for use in selectively capturing and/or separating anionic contaminants, such as chromium and arsenic oxyanions, including Cr(O), Cr(ll), Cr (III), Cr (IV), and/or Cr (VI), from other components.

### CROSS REFERENCE TO RELATED APPLICATIONS

This application claims priority under 35 U.S.C. § 119 from U.S. Provisional Application Ser. No. 63/413,171, filed Oct. 4, 2022, the disclosures of which are incorporated herein by reference in their entirety.

### STATEMENT REGARDING FEDERALLY SPONSORED RESEARCH

This invention was made with Government support under DE-SC0001015 and DE-SC0019992 awarded by the U.S. Department of Energy. The Government has certain rights in the invention.

### TECHNICAL FIELD

The disclosure provides for porous organic frameworks, and uses thereof, including for use in selectively capturing and/or separating anionic contaminants, such as chromium and arsenic oxyanions, from other components.

### BACKGROUND

Although water is the most abundant resource on Earth, it is estimated that one quarter of the global population lacks water free from contamination. The removal of micropollutants (e.g., heavy metals) from water is one of the most pressing yet technologically difficult water treatment challenges worldwide. These contaminants are often found in various water sources at trace yet toxic concentrations, alongside competing constituents of similar size and charge that are more concentrated yet relatively nontoxic (e.g., sodium chloride). For example, chromium (VI)-based oxyanions have caused immense issues related to water contamination. These heavy metals are highly mutagenic and carcinogenic to living systems yet are commonly released to the environment through industrial processes such as metallurgy, electroplating, mining, leather tanning, pigment production, and cement production. As a similarly damaging contaminant, arsenic is often present in natural Bangladeshi groundwater at concentrations much higher than the drinking water limit endorsed by the World Health Organization (10 ppb). This water contamination issue leads to an estimated 43,000 deaths per year caused by arsenic poisoning in Bangladesh alone.

Various separation methods have been proposed to achieve selective chromium and arsenic removal from water, including ion-exchange, adsorption, membrane separations, electrocoagulation, and photocatalytic degradation. Of these methods, adsorption and ion exchange are often considered as the most promising due to their low operating and capital costs, high sorption capacities, and ease of use. Adsorbents and ion-exchange resins consist of an inorganic material (e.g., zeolites, layered double hydroxides, metal oxides) or organic polymer. These materials are cost-effective but typically exhibit poor sorption kinetics, chemical and thermal stabilities, and/or recycling capabilities. Moreover, these methods typically rely on leveraging electrostatic interactions (e.g., ion-exchange) that exhibit relatively low selectivity for chromium and arsenic oxyanions over other common waterborne anions. These drawbacks necessitate the development of robust materials and methods that can more effectively achieve chromium and arsenic oxyanion separations.

### SUMMARY

Chromium and arsenic are two of the most problematic water pollutants due to their high toxicity and prevalence in various water streams. Adsorption and ion-exchange processes have been applied for the efficient removal of numerous toxic contaminants from water, including heavy metals. However, these technologies display relatively low overall performances and stabilities for chromium and arsenic oxyanion remediation. The disclosure provides for the use of porous organic frameworks as adsorbent materials that use selective chemical interactions (e.g., chelation, ion exchange, hydrogen-bonding) to selectively capture chromium and arsenic from water over other common waterborne constituents. These selective chromium and arsenic binding mechanisms are confirmed using an array of materials characterizations. Adsorption tests reveal that the porous organic frameworks of the disclosure achieve selective, near-instantaneous (equilibrium capacity within <10 s), and high-capacity binding performances for chromium and arsenic. These favorable performance metrics are uniquely enabled by the targeted chemistries, high porosities, and high functional group loadings of the porous organic frameworks disclosed herein. Cycling tests furthermore demonstrate that these adsorption methods maintain stable performance over at least 10 adsorption-desorption cycles.

The design principles of the PAFs disclosed herein can achieve the selective removal of chromium, arsenic, and boron from water using the methods of the disclosure. Porous organic frameworks appended with similar, but altered binding groups exhibit differences in chromium and arsenic adsorption behavior, revealing the importance of porous organic framework chemistry on chromium and arsenic separation performances.

The disclosure provides a method for the selective capture or separation of anionic compound(s), and/or anionic species, comprising: contacting the anionic compound(s), and/or the anionic species with a porous adsorbent that has been functionalized to comprise pendant amino-diol groups and/or amino-polyol groups. In one embodiment, the porous adsorbent is selected from porous organic polymers, porous metal particles, porous metal oxide particles, metal organic frameworks (MOF), a zeolitic organic frameworks (ZIFs), covalent organic frameworks (COFs), and porous aromatic frameworks (PAFs). In a further embodiment, the porous adsorbent is PAFs. In still a further or another embodiment, the PAFs have tetrahedral carbon nodes connected by a plurality of linkers having the structure of:

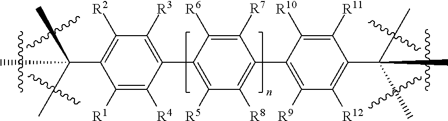

wherein, R¹-R¹² are each independently selected from H, D, an optionally substituted functional group (FG), an optionally substituted (C₁-C₉)alkyl, an optionally substituted (C₁-C₉)alkenyl, an optionally substituted (C₁-C₉) alkynyl, an optionally substituted (C₁-C₅) heteroalkyl, an optionally substituted (C₁-C₈) heteroalkenyl, an optionally substituted (C₁-C₈) heteroalkynyl, an optionally substituted aryl, an optionally substituted heterocycle, wherein at least one of R¹-R¹² is an optionally substituted FG that comprises an amino-diol group or an amino-polyol group; and n is an integer selected from 0, 1, or 2. In still another or further embodiment, the amino-diol group or the amino-polyol group has a structure selected from:

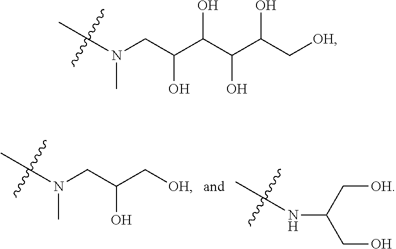

In another embodiment, the PAFs have tetrahedral carbon nodes connected by a plurality of linkers having the structure of:

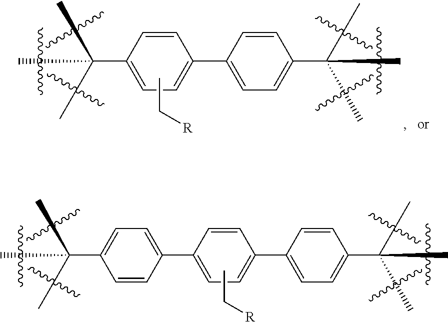

wherein, R is selected from

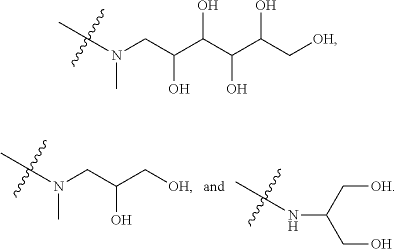

In a further embodiment, R is

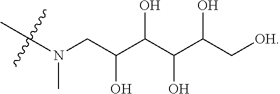 [SMILES: *N(C)CC(O)C(O)C(O)C(O)CO]

In still another or further embodiment of any of the foregoing, the adsorbent can be used to selectively capture or separate anionic compounds, and/or an anionic species comprising Cr(0), Cr(II), Cr(III), Cr(IV), and/or Cr(VI). In a further embodiment, the anionic compounds, and/or an anionic species are selected from CrO₄²⁻, HCrO₄⁻, H₂CHO₄, Cr₂O₇²⁻, HCr₂O₇⁻, and/or H₂Cr₂O₇. In still another or further embodiment of any of the foregoing embodiments, the adsorbent can be used to selectively capture or separate anionic compounds, and/or an anionic species comprising As(0), As(III), and/or As(V). In a further embodiment, the anionic compounds, and/or an anionic species are selected from AsO₄³⁻, HAsO₄²⁻, H₂AsO⁴⁻, H₃AsO₄, H₃AsO₃, H₂AsO³⁻, HAsO₃²⁻, and/or AsO₃³⁻. In still another or further embodiment, the anionic compound(s), and/or anionic species are selectively captured or separated from a fluid stream. In a further embodiment, the fluid stream is gas mixture or a water source. In still a further embodiment, the water source is groundwater, surface water, freshwater, seawater, wastewater, well water, mining water, or brackish water. In another embodiment of any of the foregoing embodiments, the anionic compound(s), and/or anionic species are waterborne oxyanions. In a further embodiment, the waterborne oxyanions are selected from vanadate, molybdate, permanganate, tungstate, antimony oxyanions, ferrate, rhenate, perrhenate, tellurate, tellurite, selenate, selenite, perxenate, carbonate, bicarbonate, phosphite, phosphate, hyposulfite, nitrite, nitrate, chlorate, sulfate, thiosulfate, acetate, perchlorate, hypochlorite, iodate, bromite, perbromate, bromate, and hypobromite.

The disclosure also provides a device comprising a porous adsorbent that has been functionalized to comprise pendant amino-diol groups and/or amino-polyol groups. In one embodiment, the porous adsorbent is selected from porous organic polymers, porous metal particles, porous metal oxide particles, metal organic frameworks (MOF), a zeolitic organic frameworks (ZIFs), covalent organic frameworks (COFs), and porous aromatic frameworks (PAFs). In another or further embodiment, the porous adsorbent is PAFs. In still a further embodiment, the PAFs have tetrahedral carbon nodes connected by a plurality of linkers having the structure of:

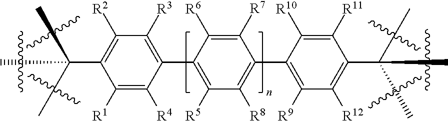

wherein, R¹-R¹² are each independently selected from H, D, an optionally substituted functional group (FG), an optionally substituted (C₁-C₉)alkyl, an optionally substituted (C₁-C₉)alkenyl, an optionally substituted (C₁-C₉) alkynyl, an optionally substituted (C₁-C₈) heteroalkyl, an optionally substituted (C₁-C₈) heteroalkenyl, an optionally substituted (C₁-C₈) heteroalkynyl, an optionally substituted aryl, an optionally substituted heterocycle, wherein at least one of R¹-R¹² is an optionally substituted FG that comprises an amino-diol group or an amino-polyol group; and n is an integer selected from 0, 1, or 2. In a further embodiment, the amino-diol group or the amino-polyol group has a structure selected from:

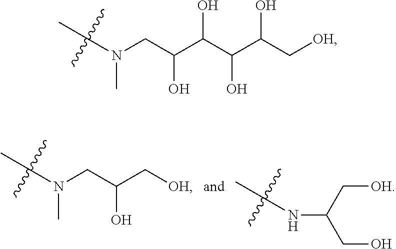

In still a further embodiment, the PAFs have tetrahedral carbon nodes connected by a plurality of linkers having the structure of:

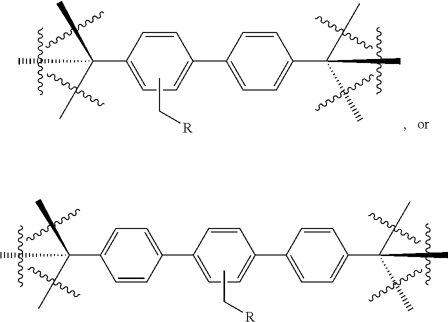

wherein, R is selected from

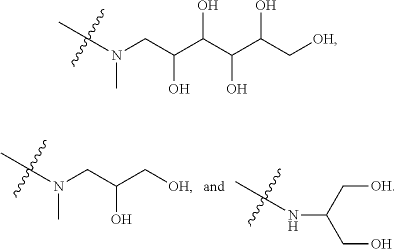

In a further embodiment, R is

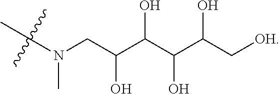 [SMILES: *N(C)CC(O)C(O)C(O)C(O)CO]

In still another or further embodiment of any of the foregoing, the adsorbent can be used to selectively capture or separate anionic compounds, and/or an anionic species comprising Cr(0), Cr(II), Cr(III), Cr(IV), and/or Cr(VI). In a further embodiment, the anionic compounds, and/or an anionic species are selected from CrO₄²⁻, HCro₄⁻, H₂CrO₄, Cr₂O₇²⁻, HCr₂O₇⁻, and/or H₂Cr₂O₇. In still another or further embodiment, the adsorbent can be used to selectively capture or separate anionic compounds, and/or an anionic species comprising As(0), As(III), and/or As(V). In a further embodiment, the anionic compounds, and/or an anionic species are selected from AsO₄³⁻, HAsO₄²⁻, H₂AsO⁴⁻, H₃AsO₄, H₃AsO₃, H₂AsO³⁻, HAsO₃²⁻, and/or AsO₃³⁻. In still another embodiment of any of the foregoing embodiments, the device is a storage device that stores the captured anionic compounds and/or anionic species. In yet another embodiment of any of the foregoing, the device is a separation or a sensor device that selectively separates, or indicates the capture, of anionic compounds and/or anionic species. In a further embodiment, the adsorbent is integrated into membranes, films, electrodes, coatings, pellets, co-polymers, porous substrates, or indicators.

The disclosure also provides a functionalized porous organic framework (PAF) having tetrahedral carbon nodes connected by linkers having the general structure of:

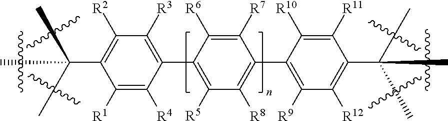

wherein, R¹-R¹² are each independently selected from H, D, an optionally substituted functional group (FG), an optionally substituted (C₁-C₉)alkyl, an optionally substituted (C₁-C₉)alkenyl, an optionally substituted (C₁-C₉) alkynyl, an optionally substituted (C₁-C₅) heteroalkyl, an optionally substituted (C₁-C₈) heteroalkenyl, an optionally substituted (C₁-C₈) heteroalkynyl, an optionally substituted cycloalkyl, an optionally substituted aryl, an optionally substituted heterocycle, wherein at least one of R¹-R¹² is an FG comprising a secondary or tertiary amine; and n is an integer selected from 0, 1, 2, or 3; wherein, the optionally substituted FG comprising a secondary and/or a tertiary amine does not have the structure of

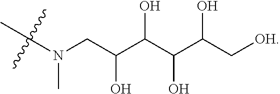 [SMILES: *N(C)CC(O)C(O)C(O)C(O)CO]

In another embodiment, at least one of R₁-R¹² is an optionally substituted FG having the structure of

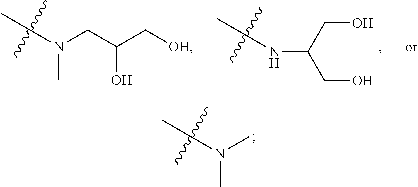

and n is an integer selected from 0, 1, 2, or 3. In another embodiment, linkers have the structure of:

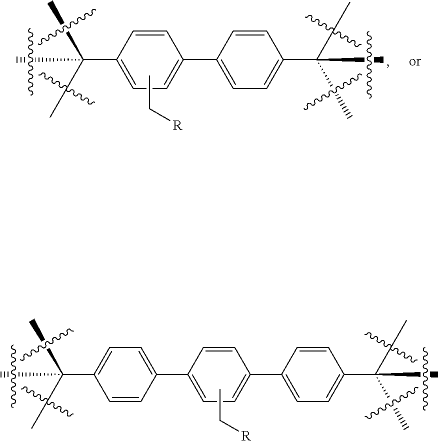

wherein, R is selected from

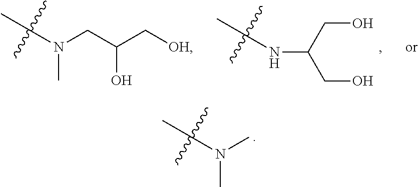

### DESCRIPTION OF DRAWINGS

FIG. 1A-B presents: (A) Schematic of functionalized PAF pores of the disclosure, where R indicates an appended functional group. (B) Exemplary functional groups appended onto PAFs of the disclosure, along with the corresponding names of the resulting materials based on the PAF-1 material that comprises biphenyl linkers. NMDG: N-methyl-D-glucamine, MAPD: 3-methylamino-1,2-propanediol, SABET: Brunauer-Emmett-Teller specific surface area, FG: functional group loading per gram of functionalized PAF material.

FIG. 2A-B presents: (A) Full FTIR spectra of PAF-1-serinol, PAF-1-MAPD, and PAF-1-CH₂Cl, along with (B) a zoom-in of the spectra from 1800-600 cm-1. The successful appendage of the serinol and MAPD functional groups were confirmed by the appearance of peaks related to 0-H (3300-3500 cm⁻¹), and C—N(˜1080 cm⁻¹), and C—O (˜1040 cm⁻¹) bonds, along with the disappearance of the peak related to the C—H wagging mode of —CH₂Cl from PAF-1-CH₂Cl (˜1260 cm⁻¹).

FIG. 3 presents nitrogen adsorption isotherms at 77 K and each corresponding BET surface area (SABET) for PAF-1 (black) and PAF-1-CH₂Cl (gray).

FIG. 4 presents nitrogen adsorption isotherms at 77 K and each corresponding BET surface area (SABET) for PAF-1-N(CH₃)₂, PAF-1-serinol, PAF-1-MAPD, and PAF-1-NMDG. The expected drop in surface area upon PAF-1 functionalization was observed, likely arising from the partial pore filling and added mass of the functional groups. Adsorption is denoted by filled symbols, while desorption is denoted by open symbols.

FIG. 5 provides thermogravimetric analysis (TGA) decomposition profiles (ramp rate: 5° C./min under flowing N₂) of the different PAF materials studied in this work.

FIG. 6 presents an FESEM image of a PAF-1-NMDG particle, which exhibits a spherical morphology and particle size of ˜200-300 nm.

FIG. 7 demonstrates chromium (VI) speciation in water as a function of solution pH for different total Cr(VI) concentrations. The gray dotted lines corresponding to neutral conditions (i.e., pSpeciation profiles were simulated using HySS software).

FIG. 8 provides Cr(VI) adsorption isotherms by PAF-1-NMDG (squares), PAF-1-MAPD (diamonds), PAF-1-serinol (circles), and PAF-1-N(CH₃)₂ (triangles). (Inset) Zoom-in of the dilute region of the isotherms. The adsorption data were fit by a single-site Langmuir model (solid lines). Error bars represent the standard deviation obtained from at least three replicate tests. The mean values obtained at each concentration are shown.

FIG. 9 provides a linearized single-site Langmuir model fits for the Cr(VI) adsorption isotherms of each tested adsorbent. Linear regression was used to fit each trendline.

FIG. 10 provides a linearized single-site Langmuir model fits for the As(V) adsorption isotherms of each tested adsorbent at pH=7 conditions. Linear regression was used to fit each trendline.

FIG. 11 provides a linearized single-site Langmuir model fits for the As(V) adsorption isotherms of each tested adsorbent at pH=4 conditions. Linear regression was used to fit each trendline.

FIG. 12 demonstrates removal of Cr(VI) by PAF-1-NMDG from DI water containing different dilute concentrations of Cr(VI). The PAF-1-NMDG dosage used was 0.5 mg/mL.

FIG. 13 presents pictures of PAF-1-NMDG, PAF-1-MAPD, PAF-1-serinol, and PAF-1-N(CH₃)₂ after Cr(VI) adsorption tests.

FIG. 14A-B presents As(V) adsorption isotherms by PAF-1-NMDG (squares), PAF-1-MAPD (diamonds), and PAF-1-N(CH₃)₂ (triangles), at (A) pH=7 and (B) pH=4 conditions as adjusted by HCl. The adsorption data were fit by a single-site Langmuir model (solid lines).

FIG. 15A-D presents Fourier-transformed EXAFS spectra and the one-shell fitting results for (A) Cr(VI)- and (B) As(V)-loaded PAF-1-NMDG. Phase shifts are not included in the displayed apparent distances. (Insets) XANES spectra for each respective sample. The gray and black dotted lines in the B inset correspond to the white line peak energies of the As(III) and As(V) oxidation states, respectively. Potential structures of (C) Cr(VI) and (D) As(V) bound to PAF-1-NMDG.

FIG. 16 provides a Cr K-edge EXAFS spectrum in k-space for Cr-loaded PAF-1-NMDG. The total fit signal (dotted line) for the Cr-loaded PAF-1-NMDG sample (solid line) is shown, along with the fit window (upper solid line).

FIG. 17 provides a As K-edge EXAFS spectrum in k-space for As-loaded PAF-1-NMDG. The total fit signal (dotted line) for the As-loaded PAF-1-NMDG sample (solid line) is shown, along with the fit window (upper solid line).

FIG. 18 provides a Cr K-edge EXAFS spectrum in k-space for Cr-loaded PAF-1-N(CH₃)₂. The total fit signal (black dotted line) for the Cr-loaded PAF-1-N(CH₃)₂ sample (gray solid line) is shown, along with the fit window (upper solid line).

FIG. 19 provides a As K-edge EXAFS spectrum in k-space for As-loaded PAF-1-N(CH₃)₂. The total fit signal (black dotted line) for the As-loaded PAF-1-N(CH₃)₂ sample (gray solid line) is shown, along with the fit window (upper solid line).

FIG. 20 provides Fourier-transformed EXAFS spectra and the one-shell fitting results for Cr(VI)-loaded PAF-1-N(CH₃)₂. Phase shifts are not included in the displayed apparent distances. (Inset) Cr K-edge XANES spectra for the sample.

FIG. 21 provides Fourier-transformed EXAFS spectra and the one-shell fitting results for As(V)-loaded PAF-1-N(CH₃)₂. Phase shifts are not included in the displayed apparent distances. (Inset) As K-edge XANES spectra for the sample. The gray and black dotted lines correspond to the white line peak energies of the As(III) and As(V) oxidation states, respectively.

FIG. 22 presents an exemplary monochelate binding complex between PAF-1-NMDG and the (left) Cr(VI) oxyanions and (right) As(V) oxyanions.

FIG. 23 presents boron adsorption isotherms by PAF-1-NMDG (squares), PAF-1-MAPD (diamonds), and PAF-1-serinol (circles). The PAF-1-NMDG adsorption data were fit using a dual-site Langmuir model, while the PAF-1-MAPD and PAF-1-serinol adsorption data were fit using a single-site Langmuir model (solid lines).

FIG. 24A-B provides adsorption kinetics for (A) Cr(VI) and (B) As(V) by PAF-1-NMDG. The initial Cr(VI) and As(V) concentrations in the testing solutions were 250 ppm. The equilibrium capacities of PAF-1-NMDG for both contaminants were reached by the first data points taken (10 s after the Cr(VI) and As(V) solutions were added). These nearly instantaneous adsorption kinetics are at attributed to the high porosities, small particle sizes, and rapid binding mechanisms of PAF-1-NMDG.

FIG. 25A-B presents two-component selectivity tests for PAF-1-NMDG at neutral conditions between (A) Cr(VI) or (B) As(V) and one type of the following competing constituents: NaCl (left most bar), NaNO₃, Na₂SO₄, or B(OH)₃ (right most). In these tests, PAF-1-NMDG was mixed with a solution containing 2 mM of Cr(VI) or As(V), along with 2 mM, 20 mM, or 200 mM of the competing constituent. q0 denotes the equilibrium uptake for PAF-1-NMDG in a solution containing only 2 mm Cr(VI) or As(V) in DI water. Error bars represent the standard deviation obtained from at least three replicate tests. The mean values obtained at each condition are shown.

FIG. 26 demonstrates adsorption of Na₂SiO₃ by PAF-1-NMDG as a function of pH. The solution pH was varied using HCl and NaOH. Starting Si concentration in each test: 10 mg/L.

FIG. 27 presents a two-component selectivity test for PAF-1-NMDG at pH=7 conditions between KH₂AsO₄ and one type of the following competing constituents: NaCl (left most bar), NaNO₃, Na₂SO₄, or B(OH), (right most bar). In these tests, PAF-1-NMDG was mixed with a solution containing 2 mM of As(V), along with 2 mM, 20 mM, or 200 mM of the competing constituent. q0 denotes the equilibrium uptake for PAF-1-NMDG in a solution containing only 2 mM Cr(VI) or As(V) in DI water.

FIG. 28 shows the uptake of Cr(VI) (left), As(V) (middle), and B (right) by PAF-1-NMDG in equimolar three-component selectivity tests at pH=7 and pH=4 conditions. The solution pH was adjusted by HCl. Error bars represent the standard deviation obtained from at least three replicate tests. The mean values obtained at each condition are shown.

FIG. 29A-B presents equimolar three-component selectivity tests by PAF-1-NMDG for K₂CrO₇, KH₂AsO₄, and B(OH)₃, at (A) pH=7 and (B) pH=4 conditions as adjusted by HCl. The concentration of each species tested was 0.2 mM (left), 2 mM (middle), or 5 mM (right). The black dotted line represents a value of unity. Error bars represent the standard deviation obtained from at least three replicate tests. The mean values obtained at each condition are shown.

FIG. 30 shows the combined Cr(VI), As(V), and B(OH)₃ uptake in the equimolar three-component selectivity adsorption tests at pH=7 (left bar) and pH=4 (Right bar) conditions. Each listed initial solute concentration corresponds to the concentration of each solute type in the testing solutions. The dotted line corresponds to the maximum uptake of PAF-1-NMDG assuming a loading of one adsorbate per NMDG functional group. Error bars represent the standard deviation obtained from at least three replicate tests. The mean values obtained at each condition are shown.

FIG. 31 demonstrates the adsorption of K₂Cr₂O₇ by PAF-1-NMDG as a function of pH. The solution pH was varied using HCl and NaOH. Starting Cr concentration in each test: 10 mg/L.

FIG. 32 demonstrates the adsorption of KH₂AsO₄ by PAF-1-NMDG as a function of pH. The solution pH was varied using HCl and NaOH. Starting As concentration in each test: 10 mg/L. Error bars represent the standard deviation obtained from three replicate tests. The mean values obtained at each pH are shown.

FIG. 33 demonstrates the regeneration and recyclability of PAF-1-NMDG for Cr(VI) (left bar) and As(V) (right bar). The adsorption solution contained 500 mg/L of Cr(VI) or As(V). Desorption was carried out using 1 M HCl and 1 M NaOH washes. No measurable loss in adsorption capacity for either toxic contaminant was observed over 10 adsorption-desorption cycles.

FIG. 34 presents photographs of PAF-1-NMDG after (left) performing Cr(VI) adsorption tests and (right) subsequently regenerating the Cr-loaded material. The material turned to its original light beige color following regeneration.

FIG. 35 presents photographs of regeneration attempts to remove loaded Cr(VI) from PAF-1-NMDG using (left) 1 M NaCl, to achieve desorption through ion-exchange only, and (right) 1 M HCl. The left image corresponds to dispersed Cr-loaded PAF-1-NMDG, while the right image corresponds to desorbed Cr(VI) in solution as H₂CrO₄.

FIG. 36 presents (left) Cr(VI) and (right) As(V) adsorption isotherms at neutral conditions by P2-NMDG. The adsorption data were fit by a single-site Langmuir model (solid lines).

FIG. 37 provides a Cr K-edge XANES spectrum for Cr-loaded P2-NMDG.

FIG. 38 presents a photograph of P2-NMDG after Cr(VI) adsorption tests. The sample displayed a green tint similar to that of Cr-loaded PAF-1-NMDG.

FIG. 39 provides a comparison of (left) As(V) and (right) Cr(VI) adsorption isotherms at neutral conditions between PAF-1-NMDG (squares) and the NMDG-functionalized commercial resin IRA743 (circles). The adsorption data were fit by a single-site Langmuir model (solid lines). Error bars represent the standard deviation obtained from at least three replicate tests. The mean values obtained at each concentration are shown.

FIG. 40 provides a comparison of (left) As(V) and (right) Cr(VI) adsorption kinetics at neutral conditions between PAF-1-NMDG (squares) and the NMDG-functionalized commercial resin IRA743 (circles). The IRA743 resin was ball-milled prior to adsorption measurements to afford particle sizes similar to those of PAF-1-NMDG for proper comparison. q0 denotes the equilibrium uptake for the adsorbents (i.e., the adsorption capacity at the end of the experiment). The insets show the adsorption data for the first ˜120 s of the experiments.

FIG. 41 presents 87 K argon adsorption isotherms for PAF-1 (circles), PAF-1-N(CH3)2 (triangles), PAF-1-MAPD (diamonds), and PAF-1-NMDG (squares). Pore size distributions of these materials were calculated using these isotherm data.

FIG. 42 provides pore size distributions of the PAF materials, as determined from the collected argon adsorption isotherms. The pore size distribution of PAF-1-serinol is expected to be highly similar to that of PAF-1-MAPD, given the similar size and functionalization degree of the MAPD and serinol groups in these materials.

### DETAILED DESCRIPTION

As used herein and in the appended claims, the singular forms “a,” “an,” and “the” include plural referents unless the context clearly dictates otherwise. Thus, for example, reference to “a functional group” includes a plurality of such functional groups and reference to “the pore” includes reference to one or more pores and equivalents thereof and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present disclosure, in connection with percentages means+13. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 58, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein an “adsorbent” refers to a molecular entity that can effectively bind and separate from a mixture of components an anionic contaminant and/or anionic species. In another embodiment an adsorbent comprises porous organic polymers, porous metal particles, porous metal oxide particles, metal organic frameworks (MOF), a zeolitic organic frameworks (ZIFs), covalent organic frameworks (COFs), or porous aromatic frameworks (PAFs). In certain embodiments, the adsorbent comprises PAFs. In certain embodiments, an adsorbent is functionalized to be selective for a particular molecular entity. In certain embodiments, the adsorbent is functionalized with a functional group disclosed herein (e.g., secondary or tertiary amine containing group). In certain embodiments, the pore of a MOF, ZIF, COF, PAF is functionalized to contain the functional group.

The term “functional group” or “FG” refers to specific groups of atoms within molecules that are responsible for the characteristic chemical reactions of those molecules. While the same functional group will undergo the same or similar chemical reaction(s) regardless of the size of the molecule it is a part of, its relative reactivity can be modified by nearby functional groups. The atoms of functional groups are linked to each other and to the rest of the molecule by covalent bonds. Examples of FG that can be used in this disclosure, include, but are not limited to, substituted or unsubstituted alkyls, substituted or unsubstituted alkenyls, substituted or unsubstituted alkynyls, substituted or unsubstituted aryls, substituted or unsubstituted hetero-alkyls, substituted or unsubstituted hetero-alkenyls, substituted or unsubstituted hetero-alkynyls, substituted or unsubstituted cycloalkyls, substituted or unsubstituted cycloalkenyls, substituted or unsubstituted hetero-aryls, substituted or unsubstituted heterocycles, halos, hydroxyls, anhydrides, carbonyls, carboxyls, carbonates, carboxylates, aldehydes, haloformyls, esters, hydroperoxy, peroxy, ethers, orthoesters, carboxamides, amines, imines, imides, azides, azos, cyanates, isocyanates, nitrates, nitriles, isonitriles, nitrosos, nitros, nitrosooxy, pyridyls, sulfhydryls, sulfides, disulfides, sulfinyls, sulfos, thiocyanates, isothiocyanates, carbonothioyls, phosphinos, phosphonos, and phosphates. In a particular embodiment, the functional group or “FG” may be optionally substituted with additional functional groups. For example, an “FG” that is an amine may be optionally substituted with one or more hydroxyls. In a certain embodiment, the “FG” comprises a secondary and/or a tertiary amine.

As used herein a “fluid” refers to a liquid or gas. The fluid can be a multicomponent fluid containing a plurality of molecular entities.

The term “porous aromatic framework” or “PAF”, refers to a framework characterized by a rigid aromatic open-framework structure constructed by covalent bonds (Ben et al., 2009, Angew. Chem., Intl Ed. 48:9457; Ren et al., 2010, Chem. Commun. 46:291; Peng et al., 2011, Dalton Trans. 40:2720; Ben et al., 2011, Energy Environ. Sci. 4:3991; Ben et al., J. Mater. Chem. 21:18208; Ren et al., J. Mater. Chem. 21:10348; Yuan et al., 2011, J. Mater. Chem. 21:13498; Zhao et al., 2011, Chem. Commun. 47:6389; Ben & Qiu, 2012, Cryst Eng Comm, DOI: 10. 1039/c2ce25409c). PAFs show high surface areas and excellent physicochemical stability, generally with long range orders and, to a certain extent, an amorphous nature. Porous aromatic frameworks lack the extended conjugation found in conjugated microporous polymers. A porous aromatic framework can have a surface area from about 50 m²/g to about 7,000 m²/g, about 80 m²/g to about 1,000 m²/g, 1,000 m²/g to about 6,000 m²/g, or about 1,500 m²/g to about 5,000 m²/g. A PAF can have a pore width of about 7 angstroms to about 30 angstroms (e.g., 10, 15, 20, 25 angstroms of any value between any of the foregoing). PAFs can have a differential pore volume of 0.02 to 0.30 cm³ g⁻¹ Å⁻¹ (e.g., 0.02, 0.05, 0.10, 0.15, 0.20, 0.25 cm³ g⁻¹ Å⁻¹ of any value between any of the foregoing values).

Various separation methods have been proposed to achieve selective chromium and arsenic removal from water, including ion-exchange, adsorption, membrane separations, electrocoagulation, and photocatalytic degradation. Of these methods, adsorption and ion exchange are often considered as the most promising due to their low operating and capital costs, high sorption capacities, and ease of use. Adsorbents and ion-exchange resins consist of an inorganic material (e.g., zeolites, layered double hydroxides, metal oxides) or organic polymer. These materials are cost-effective but typically exhibit poor sorption kinetics, chemical and thermal stabilities, and/or recycling capabilities. Moreover, these methods typically rely on leveraging electrostatic interactions (e.g., ion-exchange) that exhibit relatively low selectivity for chromium and arsenic oxyanions over other common waterborne anions. These drawbacks necessitate the development of robust materials and methods that can more effectively achieve chromium and arsenic oxyanion separations.

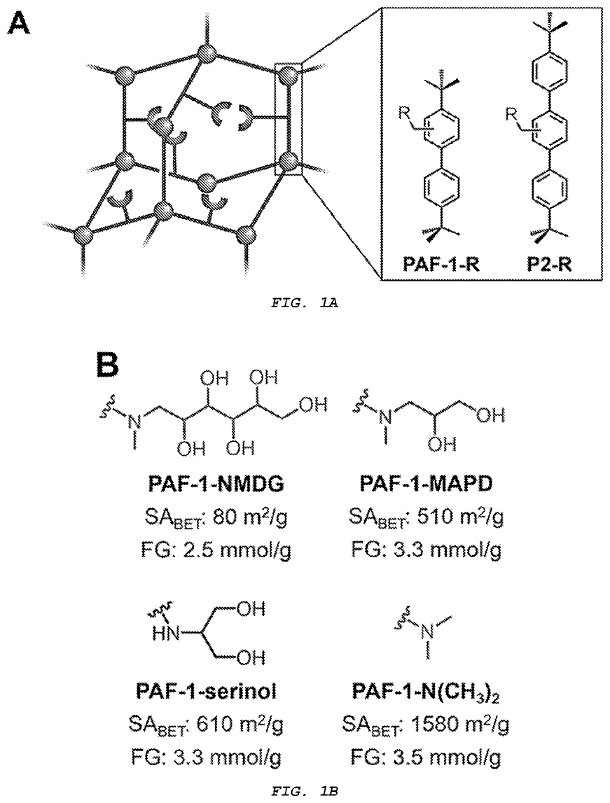

The disclosure provides a technical solution to address the limitations of existing adsorption and ion-exchange methods by developing methods based on the use of functionalized porous aromatic frameworks (PAFs). These framework materials feature targeted molecular interactions with chromium and arsenic oxyanions, leading to their high-performance removal from water. PAFs are a class of recently emerging porous polymers and porous organic frameworks that feature a high-porosity, diamondoid-like structure composed of organic nodes covalently and irreversibly coupled to aromatic linkages (see FIG. 1A). As a result, PAFs display exceptional hydrothermal and chemical stabilities, such as stability in boiling water, concentrated acids and bases, and organic solvents. The disclosure demonstrates that porous organic frameworks functionalized with the aminopolyol N-methyl-D-glucamine (NMDG) capture chromium from water through selective diol chelation interactions, with unprecedented kinetic uptake rates, high adsorption capacities, and stable cycling performance. These adsorbent materials additionally capture arsenic rapidly through selective hydrogen-bonding and ion-exchange interactions. A few reports have previously shown the use of aminopolyols and diols in macroporous polymeric materials to achieve chromium and arsenic capture, but their precise adsorption mechanisms have not been elucidated.

While aminopolyols and diols in macroporous polymeric materials achieve chromium and arsenic capture, these materials exhibit limited capacities, uptake rates, and stabilities, owing to their low porosities and synthetic tunabilities. Other material classes with higher porosities and tunabilities (e.g., metal-organic frameworks) have been reported as alternative adsorbents for chromium and arsenic oxyanion capture. However, these reported materials have typically relied on electrostatic ion-exchange interactions between the oxyanions and cationic groups on the material, leading to relatively low selectivity over other waterborne anions.

In a particular embodiment, the disclosure provides for a functionalized porous organic framework (PAF) having tetrahedral carbon nodes connected by linkers having the general structure of:

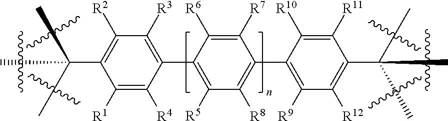

wherein,
R¹-R¹² are each independently selected from H, D, an optionally substituted functional group (FG), an optionally substituted (C₁-C₉)alkyl, an optionally substituted (C₁-C₉)alkenyl, an optionally substituted (C₁-C₉) alkynyl, an optionally substituted (C₁-C₈) heteroalkyl, an optionally substituted (C₁-C₈) heteroalkenyl, an optionally substituted (C₁-C₈) heteroalkynyl, an optionally substituted cycloalkyl, an optionally substituted aryl, an optionally substituted heterocycle, wherein at least one of R¹-R¹² is an FG comprising a secondary or tertiary amine; and
n is an integer selected from 0, 1, 2, or 3. In a further embodiment, the optionally substituted FG comprising a secondary and/or a tertiary amine does not have the structure of

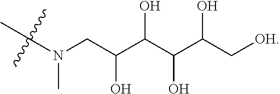 [SMILES: *N(C)CC(O)C(O)C(O)C(O)CO]

In another embodiment, the disclosure provides for a functionalized PAF having tetrahedral carbon nodes connected by linkers having the structure of:

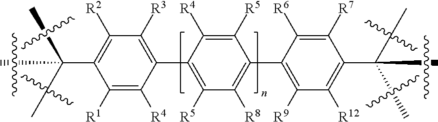

wherein,
R¹-R¹² are each independently selected from H, D, an optionally substituted functional group (FG), an optionally substituted (C₁-C₉) alkyl, an optionally substituted (C₁-C₉)alkenyl, an optionally substituted (C₁-C₉) alkynyl, an optionally substituted (C₁-C₈) heteroalkyl, an optionally substituted (C₁-C₈) heteroalkenyl, an optionally substituted (C₁-C₈) heteroalkynyl, an optionally substituted cycloalkyl, an optionally substituted aryl, an optionally substituted heterocycle, wherein at least one of R¹-R¹² is an optionally substituted FG having the structure of

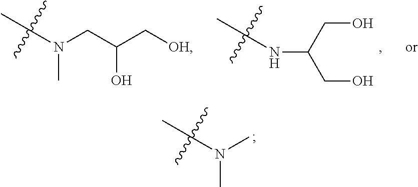

and
n is an integer selected from 0, 1, 2, or 3.

In yet another embodiment, the disclosure provides for a functionalized PAF having tetrahedral carbon nodes connected by linkers having the structure of:

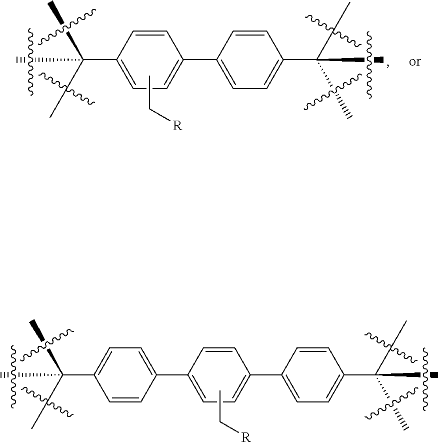

wherein,
R is selected from

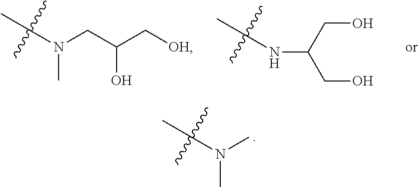

In a particular embodiment, the disclosure provides methods for the selective capture of an anionic compound, and/or an anionic species, comprising:
contacting the anionic compounds, and/or anionic species with a functionalized porous aromatic framework (PAF) having tetrahedral carbon nodes connected by a plurality of linkers having the structure of:

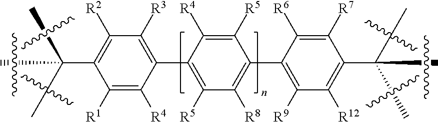

wherein,
R¹-R¹² are each independently selected from H, D, an optionally substituted functional group (FG), an optionally substituted (C₁-C₉) alkyl, an optionally substituted (C₁-C₉)alkenyl, an optionally substituted (C₁-C₉) alkynyl, an optionally substituted (C₁-C₈) heteroalkyl, an optionally substituted (C₁-C₈) heteroalkenyl, an optionally substituted (C₁-C₈) heteroalkynyl, an optionally substituted aryl, an optionally substituted heterocycle, wherein at least one of R¹-R¹² is an optionally substituted FG that comprises a secondary and/or a tertiary amine; and n is an integer selected from 0, 1, or 2. In a further embodiment, the FG comprising a secondary and/or tertiary amine comprises 1, 2, 3, 4, 5, 6, 7, 8 (or a range thereof) hydroxyl group(s). In another embodiment, at least one of R¹-R¹² is an optionally substituted FG having the structure of

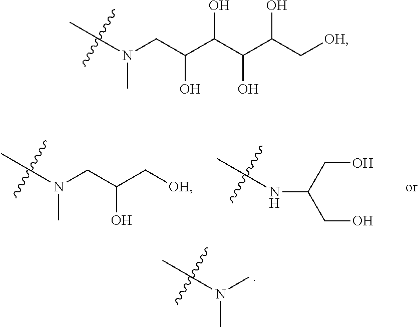

In a particular embodiment, when n is 0, then one of R¹-R⁴ is an optionally FG selected from

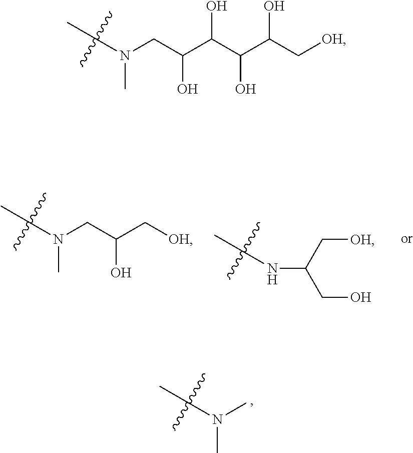

and R⁹-R¹⁰ are H. In another embodiment, when n is 1, then one of R⁵-R⁸ is optionally substituted FG selected from

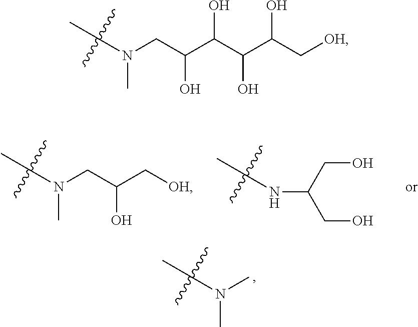

R¹-R⁴ are H, and R⁹-R¹² are H.

In a particular embodiment, the disclosure provides methods for the selective capture of an anionic compound, and/or an anionic species, comprising:
contacting the anionic compound, and/or the anionic species with a functionalized porous aromatic framework (PAF) having tetrahedral carbon nodes connected by a plurality of linkers having the structure of:

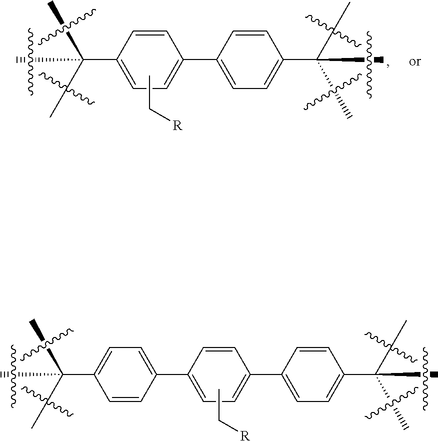

wherein,
R is selected from

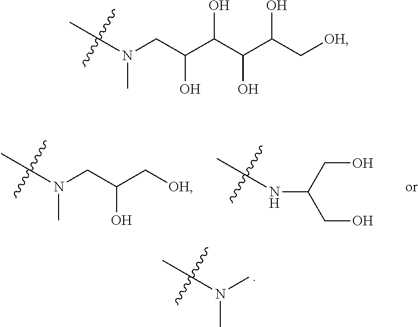

The methods of the disclosure can separate or selectively capture anionic contaminants (e.g., chromium and/or arsenic oxyanions) even in the presence of other ions and/or metals. In contrast, most ion exchangers and adsorbents developed for selective ion separations are developed for selective cation separations, rather than anion separations. In addition, current anion exchangers typically rely on functional groups that adsorb anions using ion exchange interactions only, rather than other selective interactions such as chelation and hydrogen bonding. Further, the separation mechanisms disclosed herein for isolating chromium and arsenic metals are different than the mechanisms used in previous reports for the metalloid boron. For example, the boron capture mechanism previously published did not involve ion exchange or hydrogen bonding interactions, and instead involved the filling of an originally empty p-orbital (absent in chromium and arsenic) in B(OH)₃ to bind boron.

The methods and functionalized PAFs disclosed herein, can be applied generally to various separation processes, as well as to applications that involve chromium and arsenic. Examples of potential applications and variations of the methods disclosed herein, include, but are not limited to, the following:
(1) Water remediation. The methods and functionalized PAFs of the disclosure can be used to remove chromium and/or arsenic from various types of water sources (e.g., groundwater, surface water, freshwater, seawater, wastewater, well water, mining water, brackish water).
(2) Soil and other environmental remediation. The methods and functionalized PAFs of the disclosure can be used to remove chromium and/or arsenic from solid-, liquid-, or gas-state sources.
(3) Applications and separations that involve chromium and arsenic of any oxidation state or speciation. For example, The methods and functionalized PAFs of the disclosure can be used to bind or separate Cr(0), Cr(II), Cr(III), Cr(IV), or Cr(VI)—as well as As(0), As(III), or As(V)—from mixtures. Examples of different Cr(VI) dissolved waterborne species that can be targeted include, but are not limited to, CrO₄²⁻, HCrO₄⁻, H₂CrO₄, Cr₂O₇²⁻, HCr₂O₇⁻, and H₂Cr₂O₇. Examples of different As(III) and As(V) waterborne species that can be targeted include, but are not limited to, AsO₄³⁻, HAsO₄²⁻, H₂AsO⁴⁻, H₃AsO₄, H₃AsO₃, H₂AsO³⁻, HAsO₃²⁻, and AsO₃³⁻.
(4) Separating chromium, arsenic, and boron from one another. For example, the methods and functionalized PAFs disclosed herein display chromium and boron selectivity over arsenic at neutral pH, chromium selectivity over arsenic and boron at acidic pH, and boron selectivity over chromium and arsenic at alkaline pH conditions. These selectivity differences can be leveraged to achieve chromium/arsenic/boron separations. Furthermore, charge differences to achieve selectivity using the methods and functionalized PAFs of the disclosure can be leveraged using pH swings—for example, boron and As(III) are present as neutral B(OH)₃ and H₃AsO₃, respectively, at neutral pH, while Cr(VI) and As(V) are anionic oxyanions at this pH condition.
(5) Separating chromium, arsenic, and/or boron from gaseous mixtures.
(6) Adsorption column applications. The functionalized PAFs described in this disclosure can be integrated into adsorption, chromatography, and/or separation columns.
(7) Adsorptive coating applications. Porous polymer adsorbents functionalized with aminopolyols can be coated onto surfaces (either directly or after dispersion into a polymer matrix) that can be used to capture chromium and/or arsenic.
(8) As a similar material application, other porous materials (BET surface area ≥20 m²/g), such as other porous polymers or metal-organic frameworks, that are functionalized with the NMDG functional group may also be similarly used for selective chromium and arsenic separations.
(9) In-situ reduction of chromium (VI) using the materials described in this technology. Preliminary evidence suggests that the porous organic frameworks functionalized with N-methyl-D-glucamine detailed in this disclosure may selectivity reduce aqueous chromium (VI) compounds at acidic conditions.
(10) Colorimetric detection of chromium (VI) in solution. Given that the porous organic frameworks described in this disclosure turn from a light beige to a green color upon chromium adsorption, this color change may be used to detect and/or quantify chromium (VI) in solution.
(11) Selective capture of other oxygen-rich compounds, such as silicon (e.g., Si(OH)₄ or silicate species) or other waterborne oxyanions (e.g., vanadate, molybdate, permanganate, tungstate, antimony oxyanions, ferrate, rhenate, perrhenate, tellurate, tellurite, selenate, selenite, perxenate, carbonate, bicarbonate, phosphite, phosphate, hyposulfite, nitrite, nitrate, chlorate, sulfate, thiosulfate, acetate, perchlorate, hypochlorite, iodate, bromite, perbromate, bromate, hypobromite, etc.). For example, the chelation, hydrogen-bonding, redox, and ion-exchange mechanisms used in the disclosure to achieve chromium and arsenic separation can also be leveraged to selectively remove these additional oxyanions from water or selectively bind to these oxyanions in other applications.
(12) Chromium, arsenic, and oxyanion storage. By leveraging selective chemical interactions for chromium and/or arsenic, the methods and functionalized PAFs of the disclosure can be used in devices that require the storage or immobilization of chromium and arsenic. For example, the functionalized porous organic frameworks or N-methyl-D-glucamine functional group described in the disclosure can be integrated into such devices.
(13) The porous organic frameworks described in this disclosure can be integrated into other separation and sensor devices, such as into membranes, films, electrodes, coatings, pellets, co-polymers, porous substrates, and indicators. For example, such composites can be used to achieve the chromium and arsenic separations described in detail in this disclosure. For example, membranes containing the porous organic frameworks can be used to achieve chromium and arsenic separations via facilitated transport or irreversible capture, facilitated using separation schematics such as electrodialysis, ion-capture electrodialysis, diffusion dialysis, ultrafiltration, electrofiltration, fuel cells, microfiltration, ultrafiltration, nanofiltration, reverse osmosis, capacitive deionization, and membrane capacitive deionization.
(14) The PAF-1-MAPD, PAF-1-serinol, and PAF-1-N(CH₃)₂ materials described herein (see FIG. 1) can be used generally in applications such as water remediation. The principles described in this disclosure can be used generally in various applications (e.g., water purification, atmospheric water harvesting, gas separations, membrane composites) that involve the MAPD, serinol, and N(CH₃)₂ functional groups. Other aminopolyol functional groups can also be used analogously.
(15) Using the principles described in this disclosure, other porous organic frameworks (e.g., covalent organic frameworks) and porous materials (e.g., metal-organic frameworks) can be functionalized with the N-methyl-D-glucamine functional group. These materials can be similarly used to in the various application disclosed throughout the disclosure.

### EXAMPLES

Syntheses of functionalized PAFs. The parent PAF-1 framework, which consists of tetrahedral carbon nodes connected by biphenyl linkers (see FIG. 1A), was synthesized through a Yamamoto-type Ullmann coupling reaction starting with the monomer tetrakis(4-bromophenyl) methane. The NMDG functional group was then appended onto the framework through a facile two-step route, starting with the chloromethylation of PAF-1 before the subsequent nucleophilic addition of NMDG. To investigate the effects of aminopolyol size, diol positioning, and amine anion-exchange, this synthetic strategy was also implemented to synthesize three new frameworks (PAF-1-MAPD, PAF-1-serinol, PAF-1-N(CH₃)₂) containing methylamino-1,2-propanediol (MAPD, featuring a 1,2-diol), serinol (featuring a 1,3-diol), or dimethylamine functional groups (see FIG. 1). Notably, PAF-1-N(CH₃)₂ acts as a weak base anion-exchange resin without alcohol functionalities.

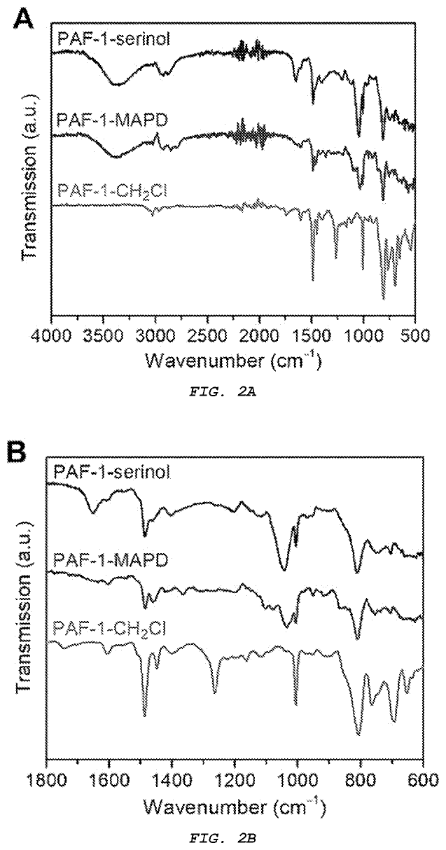

Elemental Analysis and Fourier transform infrared spectroscopy (FTIR) of the Functionalized PAFs. The successful syntheses of these materials were verified by elemental analysis, and Fourier transform infrared spectroscopy (FTIR). For each aminopolyol- or amine-functionalized material, elemental analyses revealed an increase in nitrogen content close to the values expected for a loading of one functional group per biphenyl linker. Based on nitrogen elemental analyses, the functional group loadings were calculated to be 2.50, 3.29, 3.27, and 3.51 mmol/g for PAF-1-NMDG, PAF-1-MAPD, PAF-1-serinol, and PAF-1-N(CH₃)₂, respectively (see FIG. 1B and Table 1). The FTIR spectra of PAF-1-NMDG, PAF-1-MAPD, and PAF-1-serinol showed the appearance of new peaks at 1040, 1080, and 3300-3500 (broad) cm-1, corresponding to C—O, C—N, and O—H bonds on the aminopolyols, respectively (see FIG. 2). These spectra also exhibited the disappearance of the peak at 1260 cm⁻¹, corresponding to the C—H wagging mode of —CH₂Cl on the PAF-1-CH₂Cl precursor.

TABLE 1
Functional group loadings on the functionalized PAFs,
calculated from elemental analysis results. Loadings
are provided in units of millimoles of functional group
per dry gram of overall functionalized PAF material.

| | # of functional groups | Functional group loading |
|---|---|---|
| Functionalized PAF | per biphenyl linker | (mmol/g) |
| PAF-1-CH₂Cl^{a} | 1.16 | 5.60 |
| PAF-1-NMDG^{b} | 0.91 | 2.50 |
| P2-NMDG^{b} | 1.04 | 2.36 |
| PAF-1-MAPD^{b} | 0.91 | 3.29 |
| PAF-1-serinol^{b} | 0.86 | 3.27 |
| PAF-1-N(CH₃)₂^{b} | 0.76 | 3.51 |

^{a}Loadings for PAF-1-CH₂Cl were calculated based on carbon elemental analysis.
^{b}Loadings for PAF-1-NMDG, P2-NMDG, PAF-1-MAPD, PAF-1-serinol, and PAF-1-N(CH₃)₂ were calculated based on nitrogen elemental analyses.

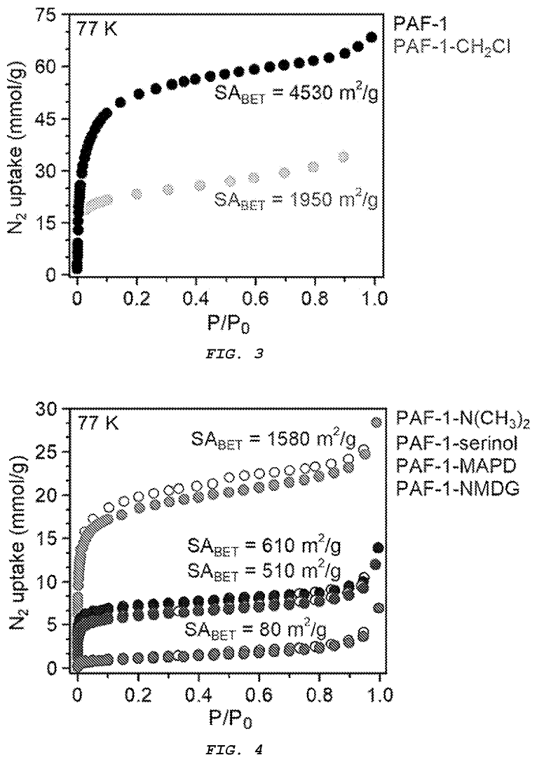

Surface area measurements of the functionalized PAFs. Nitrogen adsorption isotherms were collected at 77 K for the functionalized PAFs to determine their Brunauer-Emmett-Teller (BET) surface areas (see FIGS. 3 and 4). The obtained BET surface areas for PAF-1, PAF-1-CH₂Cl, PAF-1-N(CH₃)₂, PAF-1-serinol, PAF-1-MAPD, and PAF-1-NMDG were found to be 4530, 1950, 1580, 610, 510, and 80 m²/g, respectively. It was noted that decreases in surface area upon incorporation of functional groups into the parent PAF-1 framework are consistent with previous reports and are likely due to the partial pore filling and added mass of the functional groups. Nonetheless, the relatively high surface areas of the functionalized PAFs suggest high accessibility of the functional groups within the frameworks, as well as high porosities for these materials.

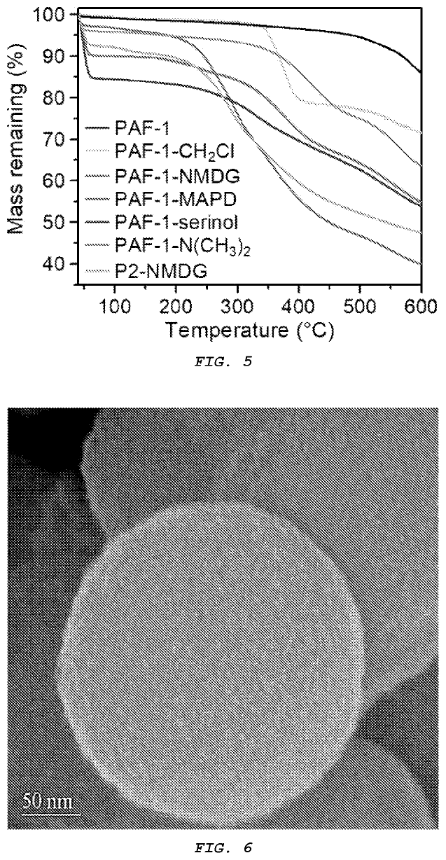

Stability measurements of the functionalized PAFs. Thermogravimetric analysis (TGA) decomposition results indicate high thermal stability of the materials (no decomposition below 200° C.), along with expected drops in mass around 200-500° C. that are consistent with the mass of the functional groups (see FIG. 5).

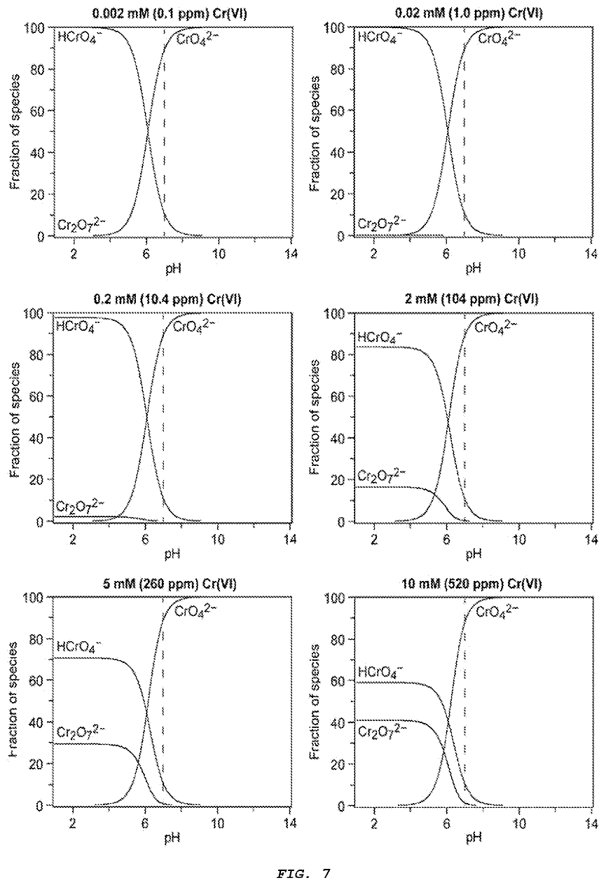

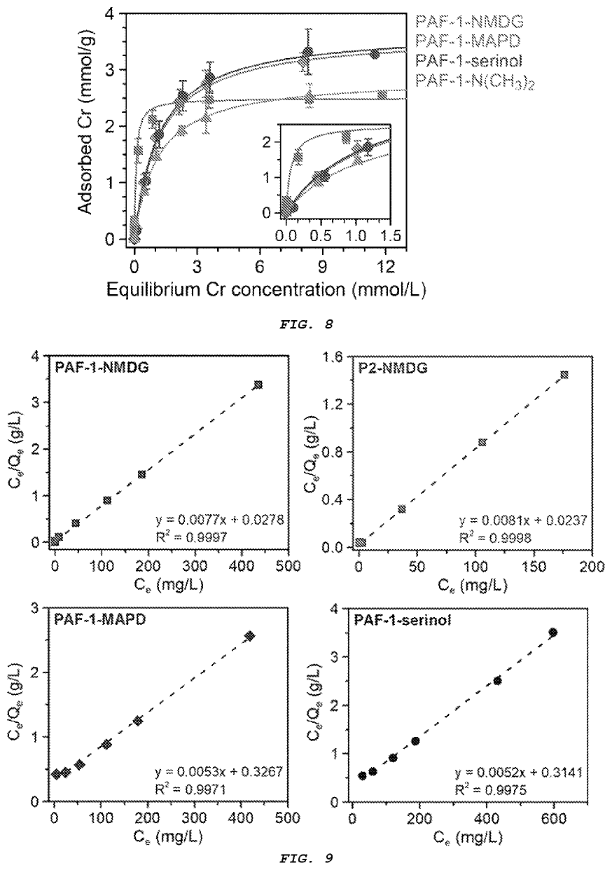

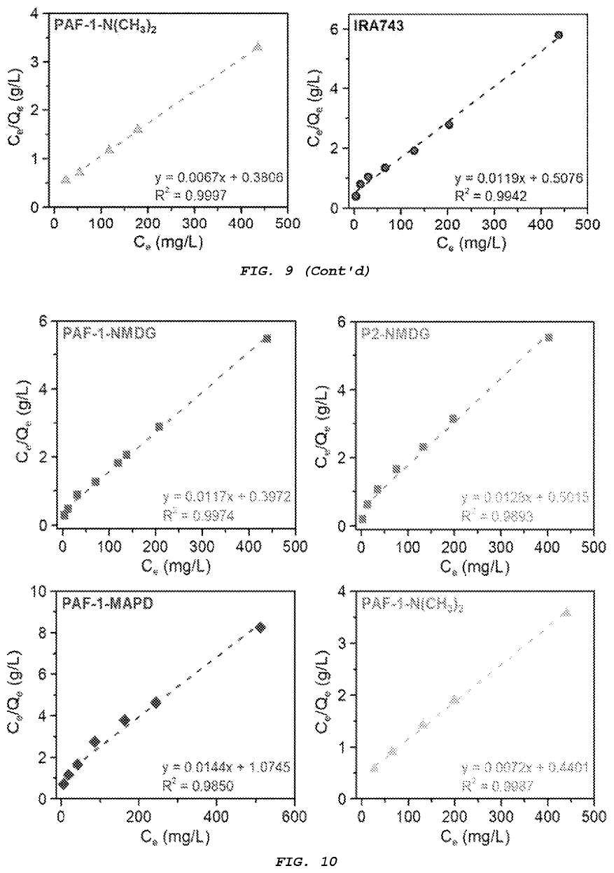

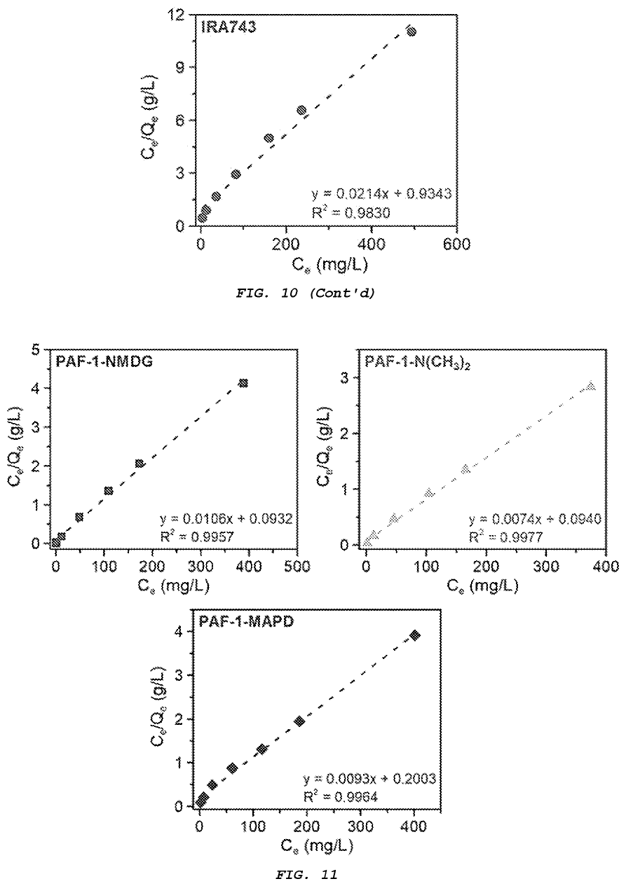

Assessing the Cr(VI) adsorption properties of the functionalized PAFs. The Cr(VI) adsorption properties of the four synthesized PAF materials was examined. Importantly, the amine on each functional group is predicted to have a pka near or above 9, causing these functional groups to be protonated at neutral pH conditions. Simulations performed using HySS software also indicate that Cr(VI) exists completely in the oxyanion state at neutral pH conditions in the measurements, with the predominant species being CrO₄²⁻ (see FIG. 7). Thus, each framework can potentially participate in anion-exchange interactions with the oxyanions, while PAF-1-NMDG, PAF-1-MAPD, and PAF-1-serinol also contain polyol groups that may additionally participate in Cr(VI) binding. The Cr(VI) adsorption isotherms were first collected and the data was fit to a single-site Langmuir model (see FIGS. 8 and 9). High Cr(VI) saturation capacities were observed for each material: 2.50, 3.63, 3.70, and 2.87 mmol/g for PAF-1-NMDG, PAF-1-MAPD, PAF-1-serinol, and PAF-1-N(CH₃)₂, respectively. These capacities suggest that the functional groups bind Cr(VI) in a ˜1:1 stoichiometric fashion (Table 2).

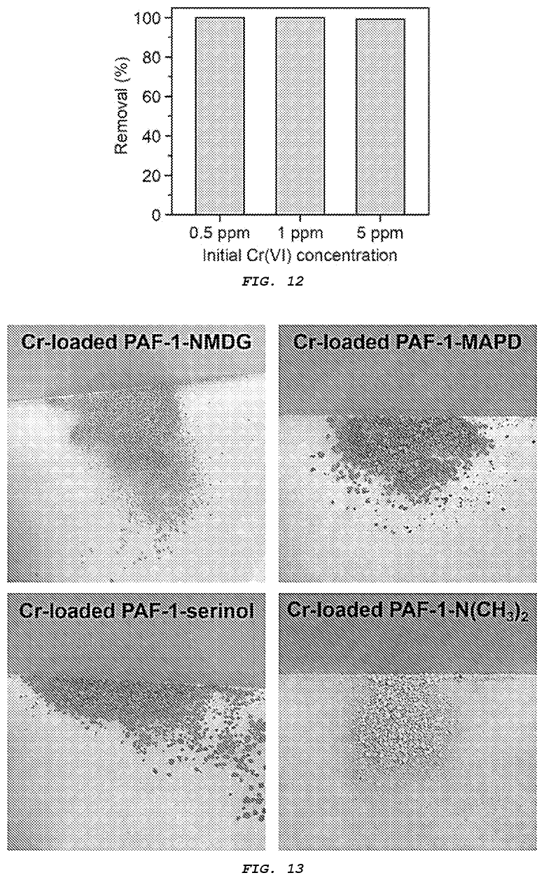

Interestingly, further inspection in the low-concentration region of these isotherms reveals that PAF-1-NMDG captures Cr(VI) much more effectively than the other frameworks at dilute Cr(VI) concentrations, indicated by the markedly steeper isotherm profile in this region (see FIG. 8 inset). Indeed, when applied to aqueous solutions containing 0.5, 1, or 5 ppm Cr(VI), PAF-1-NMDG reduces the Cr(VI) to concentrations far below the 0.1 ppm maximum contaminant level for drinking water imposed by the U.S. Environmental Protection Agency (see FIG. 12).

TABLE 2
Single-site Langmuir model fitting parameters and saturation capacities
per functional group (FG) for the obtained Cr(VI) equilibrium adsorption
isotherms. Qm is the saturation Cr(VI) adsorption capacity, KL is
the Langmuir constant, and Cr:FG is the saturation Cr adsorption
capacity per functional group loading in the adsorbent.

| | Qₘ | K_{L} | |
|---|---|---|---|
| Material | (mmol/g) | (L/mmol) | Cr:FG^{a} |
| PAF-1-NMDG | 2.50 | 14.4 | 1.00 |
| P2-NMDG | 2.39 | 17.7 | 1.01 |
| PAF-1-MAPD | 3.63 | 0.8 | 1.10 |
| PAF-1-serinol | 3.70 | 0.9 | 1.13 |
| PAF-1-N(CH₃)₂ | 2.87 | 0.9 | 0.82 |
| IRA743 | 1.62 | 1.2 | — |

^{a}Elemental analysis for IRA743 was not conducted in this report. However, a previous study found that this material possesses an NMDG loading of 2.18 mmol/g, based on nitrogen elemental analysis. According to this loading, the Cr:FG value for IRA743 would be 0.74.

The Langmuir constant (KL) obtained from the Langmuir model serves as a measure of equilibrium binding affinity. The KL value for Cr(VI) obtained for PAF-1-NMDG (14.4 L/mmol) was over an order of magnitude higher than those for PAF-1-MAPD (0.8 L/mmol), PAF-1-serinol (0.9 L/mmol), and PAF-1-N(CH₃)₂ (0.9 L/mmol) (see Table 2). These differences confirm the higher Cr(VI) binding strength toward the NMDG functional groups than to the MAPD, serinol, and dimethylamine groups. The nearly identical KL values among PAF-1-MAPD, PAF-1-serinol, and PAF-1-N(CH₃)₂ also suggest that Cr(VI) binding in each of these three frameworks occurs through a similar mechanism. Given that PAF-1-N(CH₃)₂ can only capture Cr(VI) oxyanions through anion-exchange interactions, these results suggest that PAF-1-MAPD and PAF-1-serinol also capture Cr(VI) via anion-exchange. Indeed, images of these three frameworks after Cr(VI) loading show that the frameworks gain a yellow-brown color upon adsorption (see FIG. 13), similar to the colors of Cr(VI) in solution and of Cr(VI)-loaded anion-exchange materials. In contrast, PAF-1-NMDG turns to a distinct green color upon Cr(VI) adsorption (see FIG. 13), suggesting significant changes to the CrO₄²⁻ coordination sphere upon adsorption. These findings indicate that the additional length, flexibility, and/or number of alcohols present on NMDG facilitates the stronger Cr(VI) binding by these functional groups.

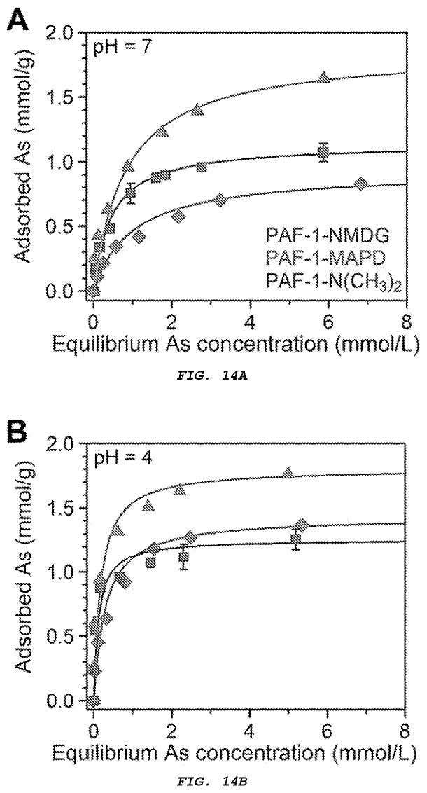

Assessing the As(V) Adsorption Properties of the Functionalized PAFs. The high Cr(VI) loadings of these frameworks encouraged studies looking at their arsenic removal capabilities. Like Cr(VI), toxic As(V) generally exists fully in the oxyanion state at neutral pH conditions, predominantly as a mixture of H₂AsO₄⁻ and HAsO₄²⁻ (pKa≈7.0). Collected As(V) adsorption isotherms and their single-site Langmuir model fits revealed saturation capacities of 1.14, 0.93, and 1.86 mmol/g for PAF-1-NMDG, PAF-1-MAPD, and PAF-1-N(CH₃)₂, respectively (see FIGS. 14A and 9; and Table 3). These capacities suggest that approximately one As(V) binds to every two NMDG or dimethylamine functional groups. The obtained KL values for PAF-1-NMDG (2.2) were approximately twice that of the values for PAF-1-MAPD (1.0) and PAF-1-N(CH₃)₂ (1.2), indicating the higher affinity of As(V) toward PAF-1-NMDG compared to the other frameworks. The similar As(V) KL values obtained for PAF-1-MAPD and PAF-1-N(CH₃)₂ suggest that PAF-1-MAPD captures As(V) through anion-exchange interactions.

TABLE 3
Single-site Langmuir model fitting parameters and saturation
capacities per functional group (FG) for the obtained As(V)
equilibrium adsorption isotherms at pH = 7 conditions.
Qm is the saturation As(V) adsorption capacity, KL is the
Langmuir constant, and As:FG is the saturation As adsorption
capacity per functional group loading in the adsorbent.

| | Qₘ | K_{L} | |
|---|---|---|---|
| Material | (mmol/g) | (L/mmol) | As:FG^{a} |
| PAF-1-NMDG | 1.14 | 2.2 | 0.46 |
| P2-NMDG | 1.04 | 1.9 | 0.44 |
| PAF-1-MAPD | 0.93 | 1.0 | 0.28 |
| PAF-1-N(CH₃)₂ | 1.86 | 1.2 | 0.53 |
| IRA743 | 0.62 | 1.7 | — |

^{a}Elemental analysis for IRA743 was not conducted in this report. However, a previous study found that this material possesses an NMDG loading of 2.18 mmol/g, based on nitrogen elemental analysis. According to this loading, the As:FG value for IRA743 would be 0.28.

Isotherms collected at pH=4 conditions showed steeper adsorption profiles for each framework but otherwise similar saturation capacities and KL differences among the frameworks (see FIGS. 14B and 10; and Table 4). At these conditions, As(V) predominantly exists as H₂AsO₄⁻, potentially leading to more favorable binding due to the additional hydrogen bonding interactions that this oxyanion species can participate in.

TABLE 4
Single-site Langmuir model fitting parameters and saturation
capacities per functional group (FG) for the obtained As(V)
equilibrium adsorption isotherms at pH = 4 conditions.
Qm is the saturation As(V) adsorption capacity, KL is the
Langmuir constant, and As:FG is the saturation As adsorption
capacity per functional group loading in the adsorbent.

| | Qₘ | K_{L} | |
|---|---|---|---|
| Material | (mmol/g) | (L/mmol) | As:FG |
| PAF-1-NMDG | 1.26 | 8.5 | 0.50 |
| PAF-1-MAPD | 1.43 | 3.5 | 0.44 |
| PAF-1-N(CH₃)₂ | 1.80 | 5.9 | 0.51 |

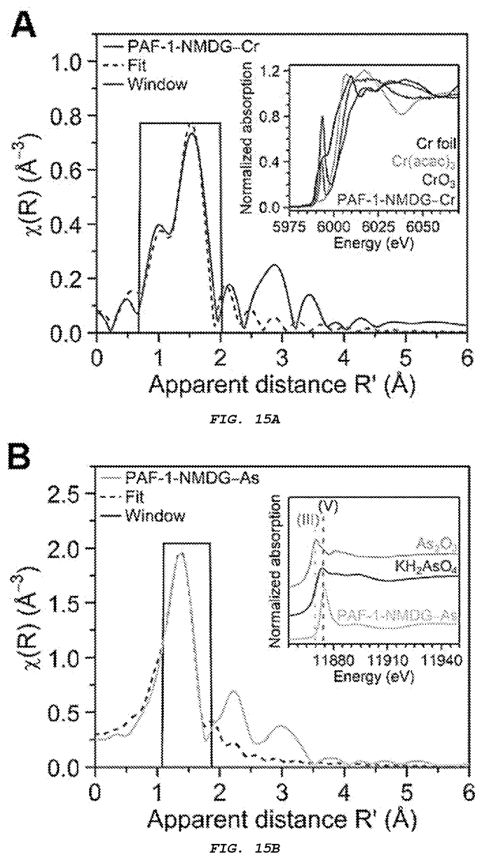

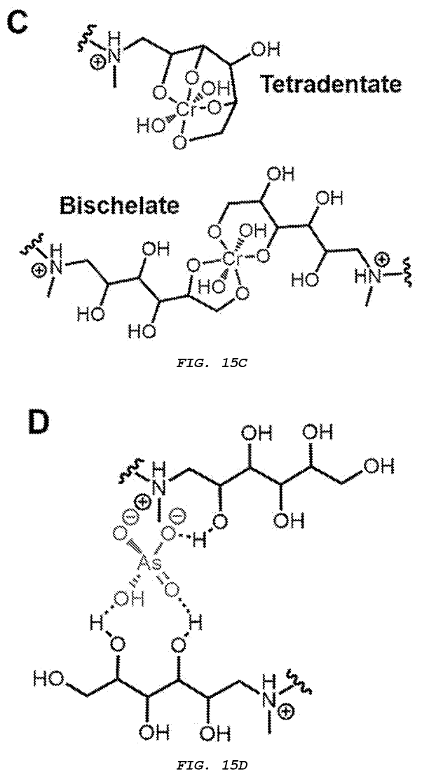

Elucidating the binding mechanisms of PAF-1-NMDG for Cr(VI) and As(V). To better elucidate the unique Cr(VI) and As(V) binding mechanisms of PAF-1-NMDG, Cr and As K-edge X-ray absorption spectroscopy (XAS) was utilized. The X-ray absorption near edge structure (XANES) spectrum for Cr-loaded PAF-1-NMDG exhibited a pre-edge feature at 5993 eV, indicative of the +6 Cr oxidation state (see FIG. 15A inset). Meanwhile, the XANES spectrum for As-loaded PAF-1-NMDG displayed a white line peak position at 11875 eV, assigned to the +5 As oxidation state (see FIG. 15B inset). Although Cr(VI) is known to oxidize alcohols under certain acidic conditions, the data suggest that the primary Cr(VI) and As(V) adsorption mechanisms by PAF-1-NMDG are not by Cr(VI) or As(V) reduction.

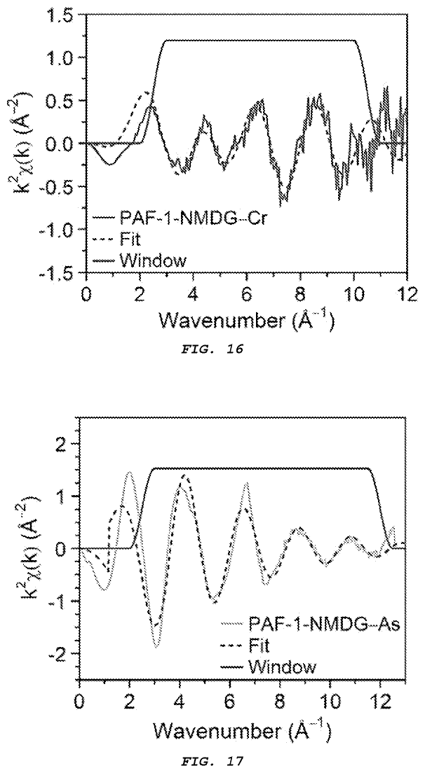

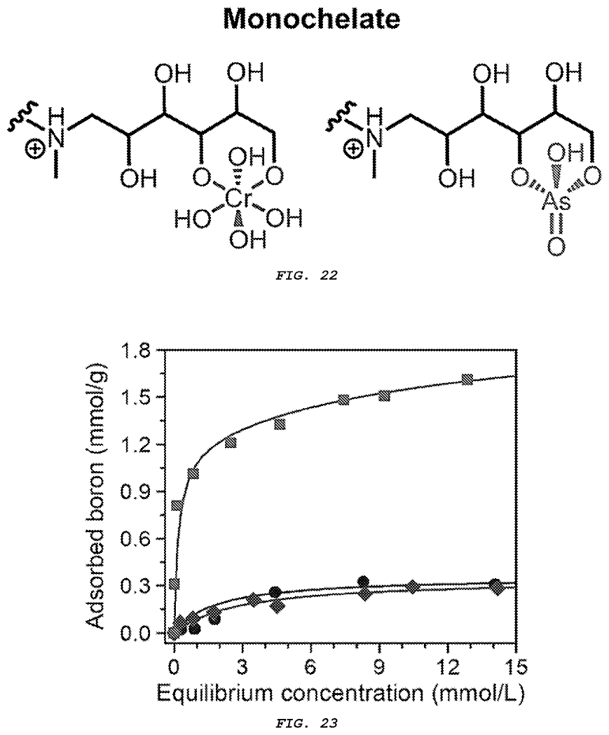

The extended X-ray absorption fine structure (EXAFS) spectra were additionally obtained and modeled for both samples (see FIG. 15A-B, and FIGS. 16-17; and Table 5). The fits indicated that Cr(VI) forms a six-coordinate complex with PAF-1-NMDG upon adsorption, consisting of four Cr—O bonds of 1.93 Å bond distance and two Cr—O bonds of 1.63 Å bond distance. The coordination number increased by two for Cr(VI) in comparison with the starting four-coordinate CrO₄²⁻ species, as confirmed of Cr(VI) coordination to the NMDG alcohol groups. Six-coordinate Cr(VI) complexes with alcohol-rich molecules is very uncommon. Potential structures of the Cr-NMDG complexes are provided in FIG. 15C (tetradentate and bischelate) and FIG. 22 (monochelate). Notably, the tetradentate complex features Cr—O bonds between Cr(VI) and four alcohols on the same NMDG functional group, while the bischelate complex exhibits Cr(VI) binding to two alcohols each on two different NMDG groups.

TABLE 5
EXAFS fitting results of the Cr- (unshaded) and As-loaded
(shaded in gray) PAF-1-NMDG and PAF-1-N(CH₃)₂ materials.

| | | | R | σ² | ΔE₀ | R-factor |
|---|---|---|---|---|---|---|
| Material | Path | N | (Å) | (Å²) | (eV) | (%) |
| PAF-1-NMDG-Cr | Cr—O | 2 | 1.63 ± 0.02 | 0.005 | −3.99 ± 7.94 | 1.8 |
| | Cr—O | 4 | 1.93 ± 0.04 | 0.005 | −3.99 ± 7.94 | 1.8 |
| PAF-1-N(CH₃)₂—Cr | Cr—O | 4 | 1.59 ± 0.01 | 0.005 | 3.94 ± 1.95 | 0.8 |
| PAF-1-NMDG-As | As—O | 4 | 1.82 ± 0.02 | 0.005 | 5.57 ± 2.43 | 0.3 |
| PAF-1-N(CH₃)₂—As | As—O | 4 | 1.70 ± 0.01 | 0.001 | 6.21 ± 2.06 | 0.5 |

Fits for the As K-edge EXAFS spectrum showed that As(V) maintains four As—O bonds upon adsorption to PAF-1-NMDG, with bond distances (1.82 Å) elongated compared to those reported in the Cambridge Crystallographic Data Centre for K₃AsO₄ (1.69 Å). Two potential As(V)-NMDG complex structures were investigated to account for the bond elongation. In the first proposed structure (see FIG. 15D), the As(V) oxyanion undergoes simultaneous anion-exchange to the protonated NMDG amine and hydrogen bonding to the polyol groups. These additional hydrogen bonding interactions may explain the improved affinity of As(V) toward PAF-1-NMDG compared to the anion-exchange-only PAF-1-N(CH₃)₂ and shorter-chain PAF-1-MAPD, as indicated by the previously described As(V) adsorption isotherms. The second proposed structure (see FIG. 22) features a monochelate mechanism, where two alcohols on one NMDG functional group chelate to one As(V) oxyanion.

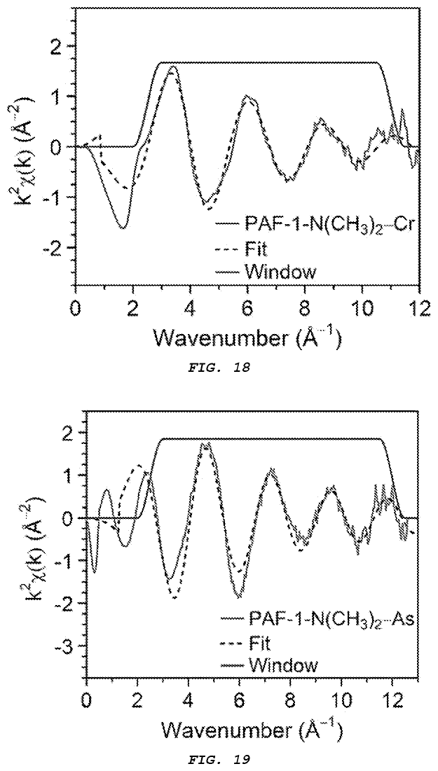

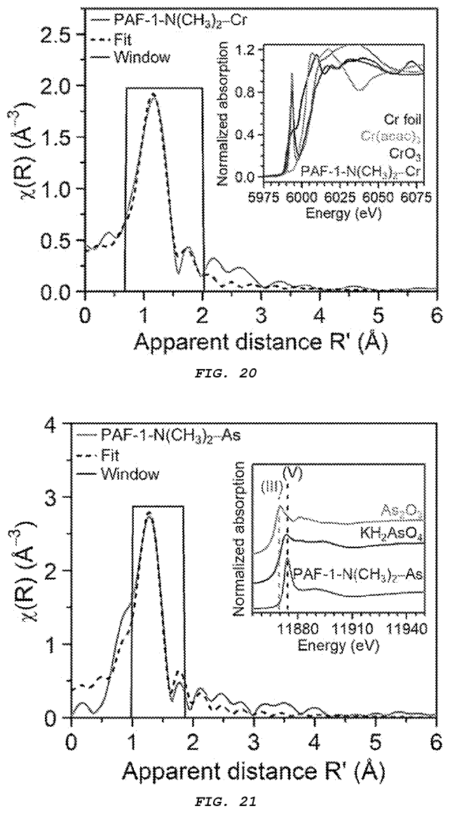

For comparison, XANES spectra was collected for Cr- and As-loaded PAF-1-N(CH₃)₂. The data likewise reveal that these adsorbates maintain their+6 and +5 oxidation states upon adsorption, respectively, as expected for anion-exchange interactions (insets of FIGS. 20 and 21). The EXAFS fits also show that the Cr maintains four Cr—O bonds of 1.59 Å bond distance upon adsorbing to PAF-1-N(CH₃)₂ (see FIGS. 18 and 20; and Table 5), highly similar to the crystallographic structure reported for Na₂CrO₄×4H₂O (Cr—O distances: 1.64 Å). Consistent with an anion-exchange mechanism, these similarities verify that the coordination sphere of Cr(VI) does not change upon CrO₄²⁻ adsorption to PAF-1-N(CH₃)₂, unlike in the case of adsorption to PAF-1-NMDG.

Investigating why shorter aminopolyols, MAPD and serinol, cannot achieve the high-affinity Cr(VI) oxyanion binding structures obtained by PAF-1-NMDG. The B(OH) 3 adsorption properties of the three frameworks were investigated. Notably, unlike for chromium or arsenic separations, the NMDG functional group is used on an industrial scale to remove boric acid from water, primarily through proposed monochelate, tetradentate, and bischelate adsorption mechanisms. The MAPD and serinol groups have not been adopted for such separations, though their lighter molecular weights could lead to higher adsorption capacities than NMDG, should they be capable of participating in the same types of chelation interactions.

Previous studies of the NMDG-B(OH)₃ adsorption mechanism revealed that B(OH)₃ adsorption is dominated by tetradentate and bischelate binding (see FIG. 15C). Meanwhile, only a small fraction of the adsorption capacity was attributed to monochelate binding mechanisms (see FIG. 22). Due to the smaller polyol sizes of MAPD and serinol, these functional groups cannot participate in tetradentate binding. However, these functional groups can theoretically participate in bischelate binding if pore spacing and functional group flexibility allow multiple functional groups to come in close enough proximity to bind to the same adsorbate molecule.

The collected B(OH)₃ isotherms show that PAF-1-NMDG achieves boron saturation capacities (2.01 mmol/g) that are roughly six times that of PAF-1-MAPD (0.33 mmol/g) and PAF-1-serinol (0.36 mmol/g) (see FIG. 23 and Table 6). These drastically lower loadings of the latter two frameworks correspond to only 1 B(OH)₃ adsorbed per every ˜10 MAPD or serinol groups on the PAFs. Given that much higher B(OH)₃ loadings are afforded through bischelate interactions, as previously explained, these data suggest that bischelate B(OH)₃ interactions—and thus presumably also bischelate Cr(VI) and As(V) interactions—do not significantly occur in PAF-1-MAPD or PAF-1-serinol. The lower Cr(VI) and As(V) binding affinities observed for PAF-1-MAPD and PAF-1-serinol can thus likely be attributed to the lack of tetradentate and bischelate binding capabilities in these frameworks.

TABLE 6
Langmuir model fitting parameters and saturation capacities
per functional group (FG) for the obtained B(OH)₃ equilibrium
adsorption isotherms. Qm, 1 and Qm, 2 are the saturation
B(OH)₃ adsorption capacities of two adsorption sites, KL,
1 and KL, 2 are the Langmuir constants of the two adsorption
sites, and B:FG is the saturation B adsorption capacity
per functional group loading in the adsorbent.

| | Q_{m, 1} | K_{L, 1} | Q_{m, 2} | K_{L, 2} | |
|---|---|---|---|---|---|
| Material | (mmol/g) | (L/mmol) | (mmol/g) | (L/mmol) | B:FG |
| PAF-1-NMDG | 1.20 | 5.4 | 0.81 | 0.08 | 0.80 |
| PAF-1-MAPD^{a} | 0.33 | 0.4 | — | — | 0.10 |
| PAF-1-serinol^{a} | 0.36 | 0.5 | — | — | 0.11 |

^{a}A single-site Langmuir model was used to fit the adsorption isotherms of the PAF-1-MAPD and PAF-1-serinol materials.

Assessing the Si(OH)₄ adsorption performance of PAF-1-NMDG as a function of pH. Silicon-based compounds pose significant industrial water treatment issues, arising from their propensity to cause scaling and the relative lack of available techniques that can selectively remove these compounds. Given the oxygen-rich speciation of waterborne silicon, it was postulated that the NMDG functional groups could potentially adsorb silicon via analogous chelating mechanisms as those observed for B(OH)₃ and Cr(VI) and As(V) oxyanions. Unfortunately, minimal Si uptake was observed at all tested pH values (pH range: 1-12). The highest Si uptake occurred at pH conditions of 9.5 and 12.0, likely because Si is largely anionic (as Si(OH)₃O⁻ and/or Si(OH)₂O₂²⁻) at these conditions, enabling some anion-exchange to the NMDG amine. It was hypothesized that the ability of B(OH)₃, Cr(VI), and As(V) to increase in coordination number (e.g., to the four-coordinate tetrahedral borate anion upon adsorption, or the six-coordinate Cr(VI) complex) plays an important role in allowing these constituents to accommodate additional coordination to the NMDG alcohol groups. This ability is lacking in Si(OH)₄.

Characterizing the Cr(VI) and As(V) removal performances of PAF-1-NMDG by measuring adsorption kinetics. Informed by the isotherm and mechanistic data provided in the previous two sections, PAF-1-NMDG was identified to be the most promising material for Cr(VI) and As(V) removal out of the four PAF-1-derived frameworks. Accordingly, the systematic characterization of the Cr(VI) and As(V) removal performances of PAF-1-NMDG was carried out, starting with adsorption kinetics measurements.

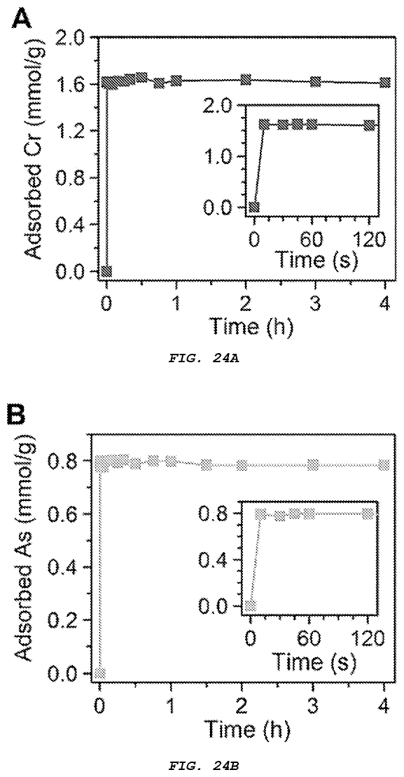

Adsorption kinetics are one of the most important properties of adsorbents, influencing factors such as throughput and required adsorbent bed sizes. The adsorption kinetics of PAF-1-NMDG was measured using separate solutions containing 250 mg/L Cr(VI) (in the form of CrO₄²⁻) or As(V) (in the form of H₂AsO⁴⁻/HAsO₄²⁻). In both cases, PAF-1-NMDG exhibited exceptionally fast kinetics: the equilibrium saturation capacities for Cr(VI) and As(V) were reached by the time of the first data point (<10 s) (see FIG. 24). The adsorption kinetics are the fastest of any reported adsorbent to date, for both chromium and arsenic. The rapid adsorption rates of PAF-1-NMDG can be attributed largely to the high porosities, indicated by high surface areas (see FIGS. 3 and 4), and small particle sizes (˜200-250 nm diameter), determined through FESEM images of FIG. 6), of this material, which minimize mass transfer resistances. Indeed, due to these properties, other functionalized PAF materials have likewise been reported to show among the fastest adsorption kinetics for their respective adsorbates.

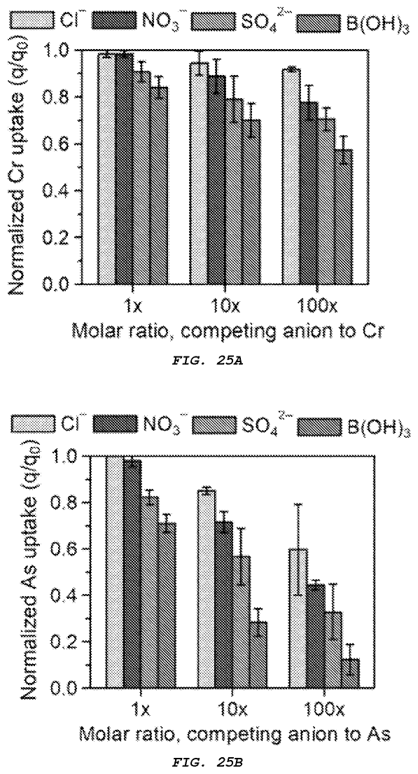

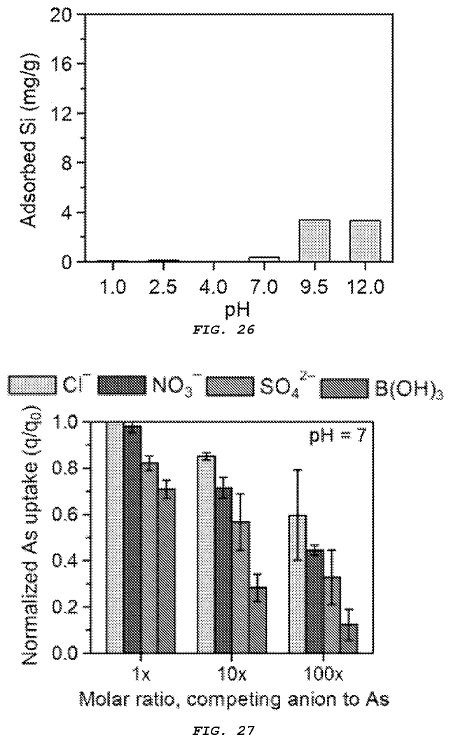

Examining the selectivity of PAF-1NMDG for Cr(VI) and As(V) two-component adsorption selectivity experiments. To understand how the unique Cr(VI) and As(V) oxyanion binding mechanisms of PAF-1-NMDG translate into selectivity over competing solutes, two-component adsorption selectivity experiments were conducted. Aqueous testing solutions were prepared by combining 2 mM of either Cr(VI) or As(V) with 2, 20, or 200 mM (i.e., 1×, 10×, or 100× higher concentrations) of one of the following common competing waterborne species: Cl⁻, NO₃⁻, SO₄²⁻, or B(OH)₃. Adsorption experiments were then conducted by mixing these solutions with PAF-1-NMDG. As displayed in FIG. 25, the measured PAF-1-NMDG Cr(VI) and As(V) adsorption capacities for each solution (q) were compared to the adsorption capacities measured for solutions devoid of competing constituents (q0) (i.e., for solutions containing only 2 mM Cr(VI) or As(V) in DI water). Strong selectivity for As(V) and especially Cr(VI) by PAF-1-NMDG was observed in these measurements. For example, the majority of the Cr(VI) capacity was maintained even in the presence of 100× higher concentrations of the competing solutes.

However, these selectivity tests also reveal that Cr(VI) and As(V) adsorption is most negatively affected by competing solutes in the following order: B(OH)₃>SO₄²⁻>NO₃⁻>Cl⁻. The measurements also demonstrated that selectivity for Cr(VI) over other competing solutes is significantly higher than the As(V) selectivities, consistent with the unique polyol chelation properties confirmed only for Cr(VI), along with the steeper adsorption isotherm profile observed for Cr(VI) (see FIGS. 8, 14 and 15). Specifically, PAF-1-NMDG showed excellent selectivity over Cl⁻, which is the most abundant anion in many water streams. For example, approximately no Cr(VI) or As(V) capacity was lost in the presence of equimolar Cl—, and only 88 loss in Cr(VI) capacity was observed even in the presence of 200 mM Cl⁻. The additional loss in Cr(VI) and As(V) capacity observed in the presence of NO₃⁻ and SO₄²⁻ was observed likely because the oxygen components of these competing anions can hydrogen-bond with the polyol functionalities, partially blocking Cr(VI) chelation and As(V) ion-exchange and hydrogen bonding with NMDG. Between these two competing anions, SO₄²⁻ decreases Cr(VI) and As(V) capacity more, likely because SO₄²⁻ possesses a higher charge and number of oxygen atoms, strengthening ion-exchange and hydrogen bonding interactions with NMDG. The presence of B(OH)₃ decreased Cr(VI) and As(V) capacity the most of any competing solute due to the known ability of PAF-1-NMDG to chelate to B(OH)₃, as previously discussed.

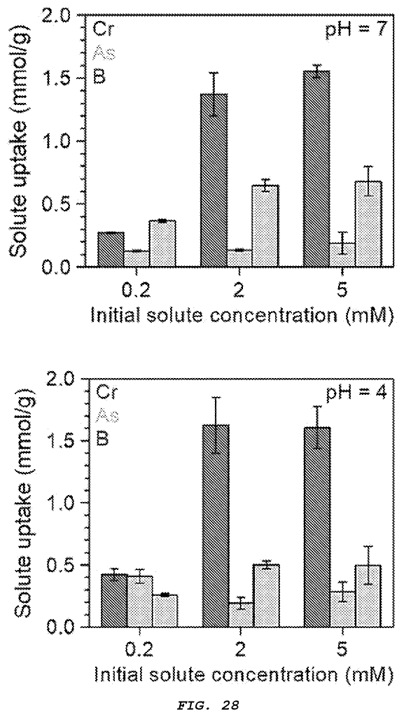

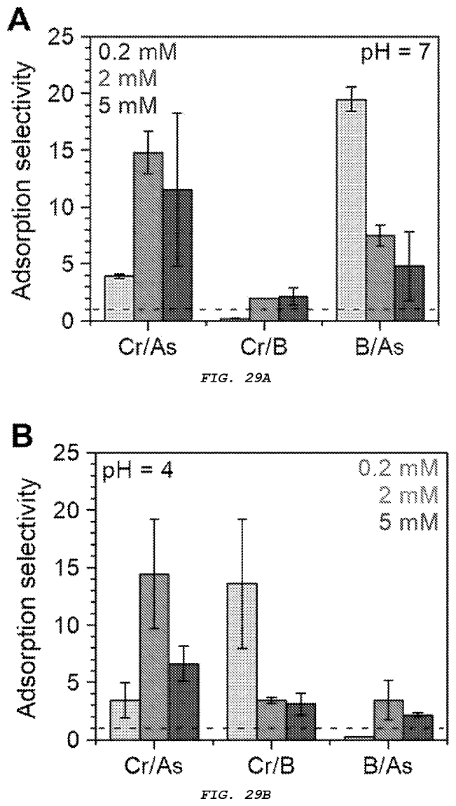

Further examining the selectivity of PAF-1NMDG for Cr(VI) and As(V) three-component adsorption selectivity experiments. Since PAF-1-NMDG can selectively capture Cr(VI), As(V), and B(OH)₃, three-component selectivity tests were performed to better understand the PAF-1-NMDG adsorption selectivities between each solute. Aqueous solutions containing equimolar concentrations of these three solutes were tested. The concentration of each solute was varied as 0.2, 2, or 5 mM to also probe concentration effects. Adsorption capacities in these experiments are provided in FIG. 28, while FIG. 29 displays the data in terms of selectivity. The results show that PAF-1-NMDG exhibits selectivity at neutral conditions in the approximate order: B(OH)₃≈Cr(VI)>As(V) (see FIG. 29A). In detail, the Cr/As selectivity ranged from 3.9-14.8 in the tested conditions, while the B/As selectivity ranged from 4.8-19.5. The higher selectivity achieved for Cr(VI) and B(OH)₃ over As(V) stems from the selective polyol chelation mechanisms observed for Cr(VI) and B(OH)₃, which leads to an increase in coordination number upon adsorption, whereas As(V) does not increase its number of bonds upon NMDG adsorption. Although the measured Cr/B selectivities approach unity at each tested condition, it is worth noting that higher Cr/B selectivity (up to 2.2) is observed at higher solute concentrations, while lower Cr/B selectivity (0.20) occurs at lower (0.2 mM) solute concentrations.

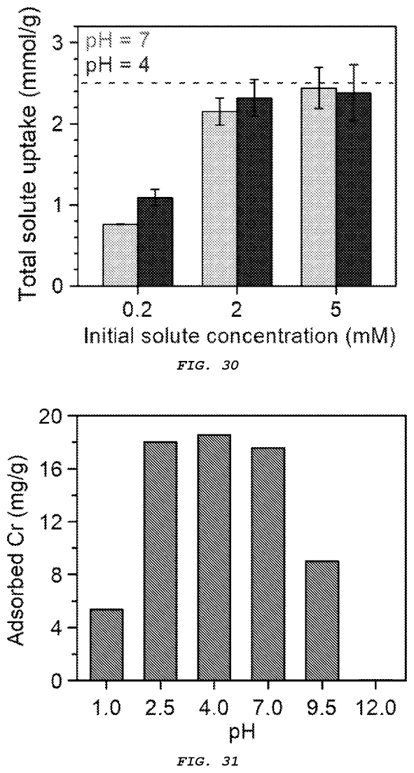

Additionally, the total combined solute adsorption capacity by PAF-1-NMDG plateaus to a ˜1:1 solute-to-NMDG ratio in these three-component experiments (see FIG. 30). This result suggests that each NMDG group cannot accommodate more than one solute at a time on average. For example, even if one NMDG polyol group chelates to one B(OH)₃ or CrO₄²⁻ molecule, the amine on that NMDG group cannot also be used to capture an additional Cr(VI) or As(V) oxyanion via ion-exchange. This behavior corroborates previously proposed B(OH)₃ binding mechanisms that suggest that the amine is intrinsically linked to the polyol chelation, due to the ability of the amine to facilitate necessary proton transfer during the binding reaction between the NMDG O—H groups and the B(OH)₃ (or, here, CrO₄²⁻) solute.

Assessing pH dependency of PAF-1-NMDG for Cr(VI) and As(V) binding. The aforementioned three-component selectivity tests were performed for PAF-1-NMDG under pH=4 conditions. Selectivities from these experiments are given in FIG. 29B, while FIG. 28 displays the data in terms of adsorption capacity. The following selectivity order under these pH=4 conditions differs from the pH=7 data: Cr(VI)>B(OH)₃≈As(V). Thus, adjusting the solution pH to slightly more acidic conditions (e.g., pH=4) can be utilized to improve Cr(VI) and As(V) selectivity further. In detail, the Cr/As selectivity ranged from 3.4-14.5 in the tested pH=4 conditions, while the Cr/B selectivity ranged from 3.1-13.6. Although the measured B/As selectivities approach unity at each tested concentration, it is worth noting that higher B/As selectivity (up to 3.5) is observed at higher solute concentrations, while lower B/As selectivity (0.25) is obtained at lower (0.2 mM) solute concentrations. Acidic conditions are expected to improve Cr(VI) chelation to PAF-1-NMDG because HCrO₄⁻ (the predominant Cr(VI) species at pH=4; see FIG. 7) must become protonated during the polyol chelation (see FIG. 15C), unlike B(OH) 3 during chelation. The improved As(V) selectivity corroborates the stronger As(V) affinity observed in pH=4 adsorption isotherms (see FIG. 14), likely due to the additional hydrogen bonding interactions and/or improved ease in chelating interactions, as previously discussed.

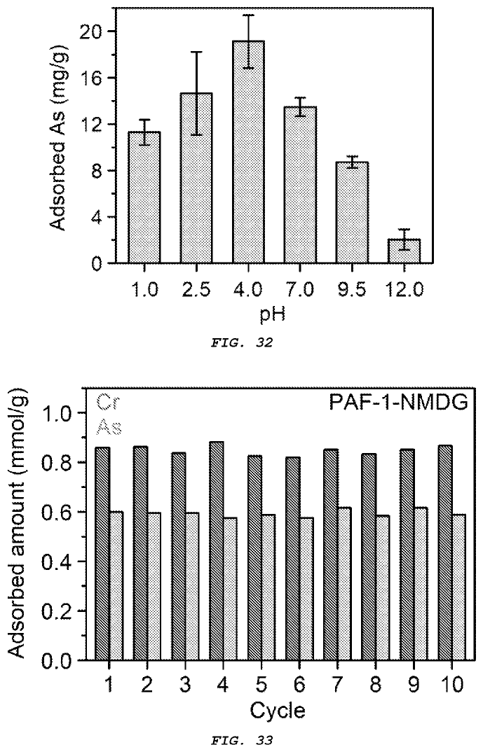

The improved Cr(VI) and As(V) selectivities observed under mildly acidic conditions also agree with other pH dependency tests that were performed. For example, two-component selectivity measurements also exhibited improved As(V) selectivity by PAF-1-NMDG over other competing solutes (Cl⁻, NO₃⁻, SO₄²⁻, B(OH)₃) at pH=4 conditions compared to at pH=7 conditions (see FIG. 27). The Cr(VI) and As(V) adsorption capacities of PAF-1-NMDG was also tested as a function of pH ranging from 1-12, using solutions containing 10 mg/L of either Cr(VI) or As(V) (see FIGS. 31-32). Maximum capacities were attained under pH=4 conditions for both adsorbates.

Notably, minimal capacities were achieved under pH=12 conditions. At these basic conditions, the NMDG groups (pKa≈9.6) are not expected to readily facilitate proton transfer in aqueous solution, and repulsive interactions between the oxyanions and deprotonated NMDG hydroxyl groups may additionally occur. These effects were similarly observed for B(OH)₃ capture under basic conditions.

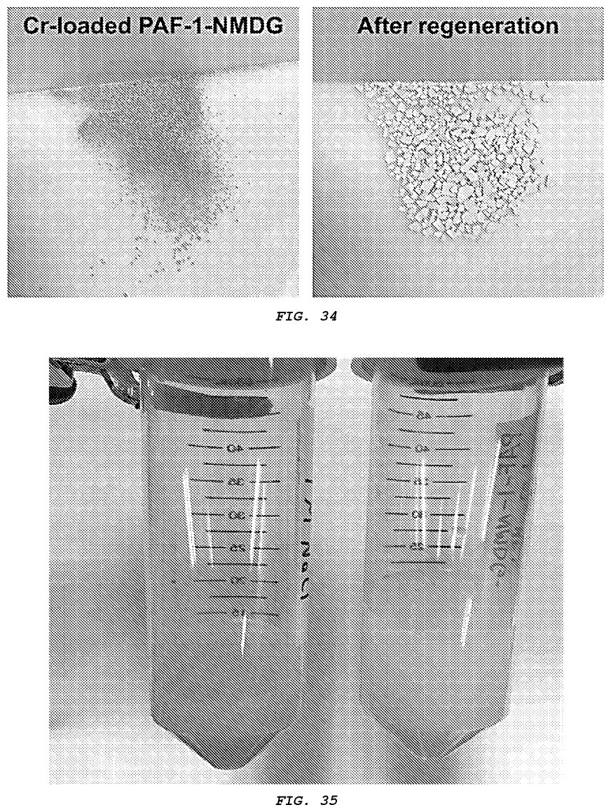

Testing the regeneration and recyclability of PAF-1-NMDG for Cr(VI) and AS (V). To maximize their lifetime and reduce capital costs, adsorbents must be capable of achieving regeneration over numerous cycles without facing significant performance loss or degradation. To this point, Cr(VI) and As(V) adsorption-desorption performances of PAF-1-NMDG were tested over 10 cycles (see FIG. 33). Inspired by the desorption protocols used for desorbing chelated boron from NMDG groups relatively mild acid and base washes (using 1 M HCl and 1 M NaOH) were applied for the desorption of Cr(VI) and As(V) from PAF-1-NMDG. Remarkably, the adsorption capacity of PAF-1-NMDG for both Cr(VI) and As(V) remained constant even after 10 cycles. As further verification of this successful recycling, Cr-loaded PAF1-NMDG displayed the characteristic green tint after every adsorption cycle, while reverting to its original light beige color after every desorption cycle (see FIG. 33). Notably, regeneration attempts using 1 M NaCl instead of 1 M HCl did not lead to noticeable Cr(VI) desorption from Cr-loaded PAF-1-NMDG (see FIG. 35). This finding validates the importance of acidic conditions to achieve desorption and further confirms that anion-exchange mechanisms are not primarily responsible for dilute Cr(VI) adsorption in PAF-1-NMDG. These results showcase the extraordinary reusability and stability of the PAF adsorbents.

Determining whether the binding strategies utilized by PAF-1-NMDG for Cr(VI) and As(V) can be replicated in analogous materials. To see if the Cr(VI) and As(V) binding strategies utilized by PAF-1-NMDG could be applied more generally to other PAF materials, an analogous material (P2-NMDG, see FIG. 1A) was synthesized having terphenyl linkers in the PAF backbone rather than biphenyl linkers.

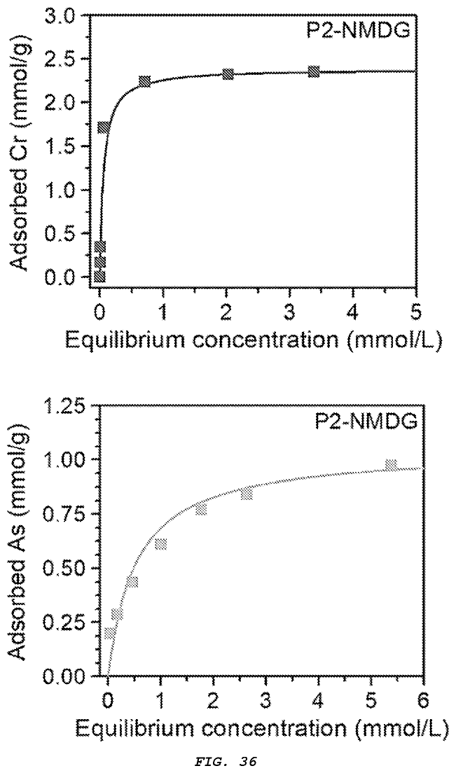

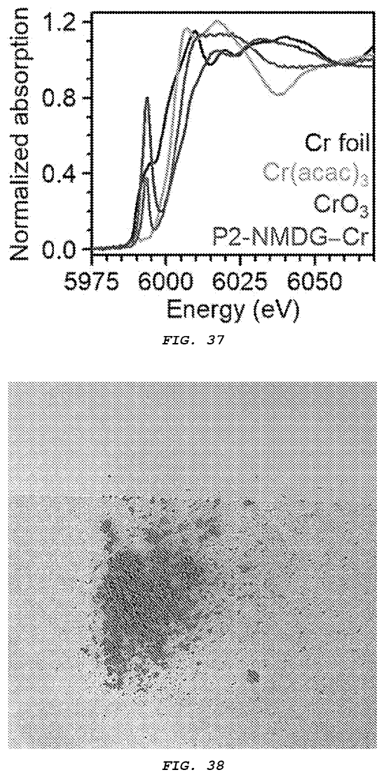

Characterization data obtained for this material were similar to those of PAF-1-NMDG. For example, consistent with previous reports, elemental analyses demonstrated that P2-NMDG possesses an NMDG loading of 2.36 mmol/g (see Table 2) and a similar TGA decomposition profile (see FIG. 5). Adsorption isotherms for P2-NMDG showed highly similar Cr(VI) and As(V) properties compared to PAF-1-NMDG: ˜1:1 NMDG:Cr and ˜2:1 NMDG:As loadings and high KL values of 17.7 and 1.9 L/mmol for Cr(VI) and As(V), respectively (see FIGS. 9, 10 and 36; and Tables 2 and 3). Adsorption characteristics also suggested that P2-NMDG binds to these adsorbates in similar fashions to those by PAF-1-NMDG. To this point, Cr-loaded P2-NMDG displayed a green color and Cr K-edge XANES spectrum (see FIG. 37) practically identical to those of Cr-loaded PAF-1-NMDG (see FIG. 15A).

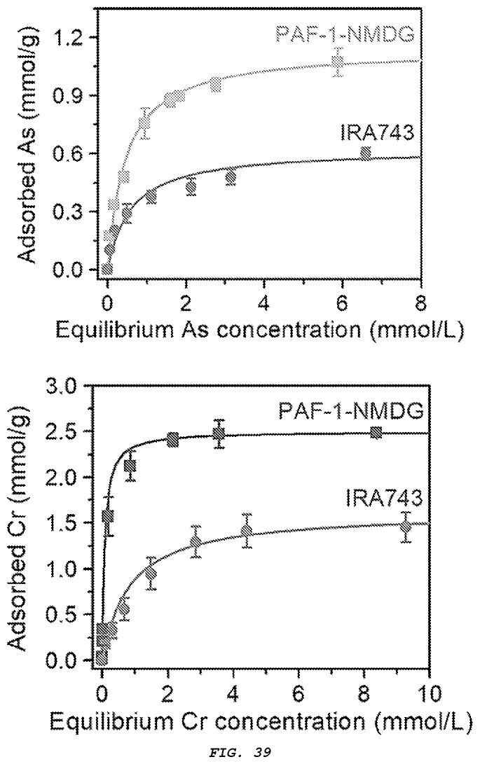

Assessing the adsorption performances of PAF-1-NMDG in comparison with Amberlite IRA743. To provide context for the adsorption performances of PAF-1-NMDG, the adsorption performances of Amberlite IRA743 was investigated for comparison. This material is a commercial resin made up of NMDG-functionalized poly(styrene-divinylbenzene). Adsorption isotherms of IRA743 for Cr(VI) and As(V) exhibited saturation capacities that were only 64.6% and 54.6% that of the Cr(VI) and As(V) saturation capacities, respectively, for PAF-1-NMDG (see FIGS. 9, 10, and 39; and Tables 2 and 3). The higher loadings of PAF-1-NMDG are enabled by the higher NMDG loadings in these high-surface-area PAF materials (see Tables 2 and 3). The KL values of IRA743 obtained for Cr(VI) (1.2) and As(V) (1.7) were also significantly lower than those for PAF-1-NMDG, suggesting more favorable binding by PAF-1-NMDG despite having the same NMDG functionality as IRA743. It was found that PAF-1-NMDG yields higher KL values for B(OH) 3 adsorption compared to those obtained for IRA 743. Given the lower synthetic control of the IRA743 pores compared to the highly tunable PAF micropores, it was postulated that spacing between NMDG functional groups may be closer and more favorable in the PAF-1-NMDG materials, allowing strong bischelate interactions. Differences in the swelling behaviors of these materials may also contribute to the differences in their binding strengths.

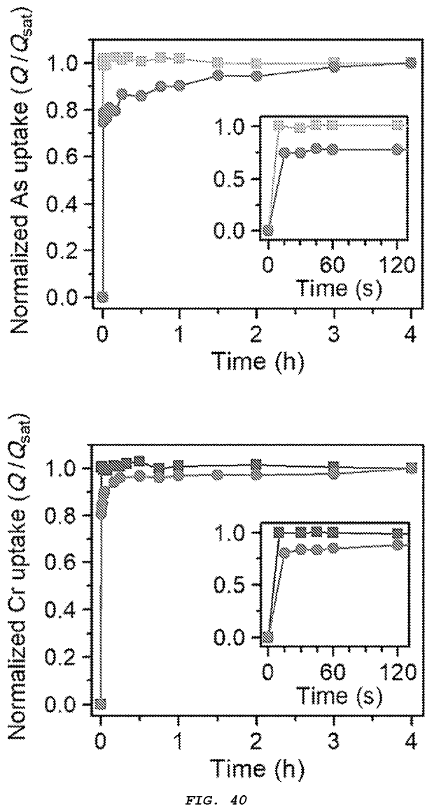

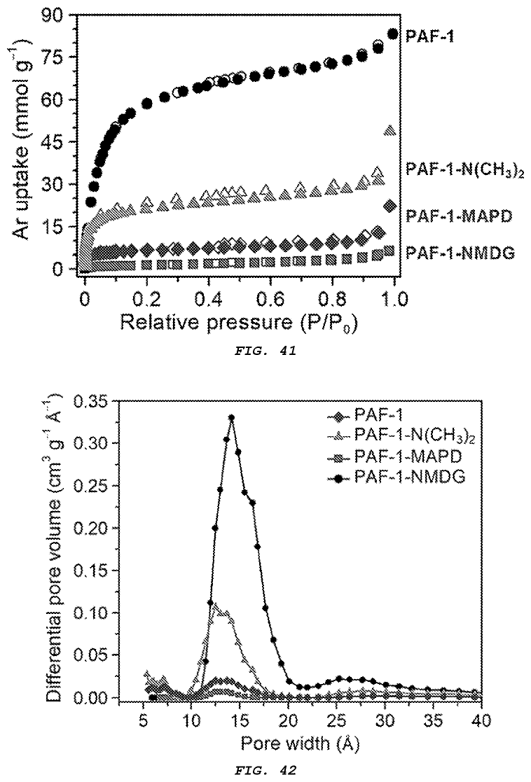

Assessing the adsorption kinetics of PAF-1-NMDG in comparison with Amberlite IRA743. The Cr(VI) and As(V) adsorption kinetics of Amberlite IRA743, was tested using the same kinetics testing protocols as used for PAF-1-NMDG. The resin was first ball-milled to afford smaller particle sizes similar to those of PAF-1-NMDG, to diminish kinetic effects from particle size differences. For both oxyanions, the ball-milled IRA743 resin displayed markedly slower adsorption kinetics compared to PAF-1-NMDG (see FIG. 40). While PAF-1-NMDG reached equilibrium capacity within 10 s, IRA743 did not reach full equilibrium capacity until after 4 h, although by 30 min the resin did reach nearly 97% and 86% of the equilibrium capacities for Cr(VI) and As(V), respectively. The drastically faster adsorption kinetics for PAF-1-NMDG can be attributed to its much higher porosity, as previously described.

PAF-1-NMDG adsorption performance in comparison to leading adsorbents. Tables 7 and 8 compare the Cr(VI) and As(V) adsorption performances, respectively, of PAF-1-NMDG more generally to the performances of other leading sorbents. As previously mentioned, PAF-1-NMDG exhibits the fastest Cr(VI) and As(V) adsorption kinetics of any reported material to date, while maintaining high capacities on par with some of the highest capacity materials. For example, other materials that achieve even higher capacities often take hours—even days—to reach their high equilibrium capacities. Unlike nearly all other materials, PAF-1-NMDG can additionally be recycled numerous times without any measurable loss in performance. It is worth noting that the majority of the other reports did not provide recyclability data, making further comparisons challenging. Importantly, with the exception of metal oxide materials used for As(V) removal, the vast majority of reported Cr(VI) and As(V) adsorbents also rely on simple ion-exchange mechanisms, which often face low selectivities not described in Tables 7 and 8. In contrast, PAF-1-NMDG features unique chelating capabilities.

TABLE 7
Comparison of the binding mechanism, percentage of adsorption capacity maintained
upon adsorption-desorption regeneration cycles, saturation adsorption capacity,
and kinetic uptake rates for Cr(VI) among PAF-1-NMDG and reported state-of-
the-art adsorbents. The pH condition implemented for each reported value is
also included. The vast majority of reported adsorbents rely on ion-exchange
interactions, which can also lead to adsorption of undesired competing anions
and thus selectivity issues not shown in this table.

| | | | | | Time to Reach | | |
|---|---|---|---|---|---|---|---|
| | | | | | Saturation | | |
| | | | | | (% of | | |
| | Primary | | | Cr(VI) | equilibrium | | |
| | Binding | Regenerability | | Capacity | capacity reached | | |
| Material | Mechanism | (cycles) | | (mg/g) | at time) | | pH |
| PAF-1-NMDG | Chelation | 100% | (10) | 130 | <10 s | (100) | 7 |
| IRA743 Resin | Chelation | — | | 84 | 4 h | (100) | 7 |
| Crosslinked Chitosan | Ion-exchange | 100% | (5) | 325 | 24 h | (100) | 2 |
| Chitosan Grafted | Ion-exchange | 85% | (10) | 104 | 7 h | (100) | 2 |
| Graphene Oxide | | | | | | | |
| Ionic Liquid | Ion-exchange | — | | 196 | — | | 1.5 |
| Impregnated | | | | | | | |
| Amberlite XAD-4 | | | | | | | |
| Amine-Functionalized | Ion-exchange, | — | | 159 | — | | 3 |
| Corn Stalk | reduction | | | | | | |
| Chitosan-Cyclodextrin | Ion-exchange, | 95% | (5) | 68 | — | | 3 |
| Grafted Graphene | reduction | | | | | | |
| Oxide | | | | | | | |
| Tulsion A-27 (MP) | Ion-exchange | — | | 57 | — | | 5.5 |
| Cereal By-Product | Ion-exchange | — | | 132 | — | | 6 |
| Carbon | | | | | | | |
| Bacterial Cellulose- | Ion-exchange | 92% | (5) | 230 | — | | 6 |
| Attapulgite | | | | | | | |
| MOR-1-HA | Ion-exchange | — | | 117 | 3 min | (97.5) | 3 |
| MOF-808 | Ion-exchange, | 95% | (1) | 50 | — | | 4.6 |
| | hydrogen | | | | | | |
| | bonding | | | | | | |
| ABT•2ClO₄ | Ion-exchange | — | | 51 | 6 h | (90) | — |
| ZJU-101 | Ion-exchange | — | | 118 | 10 min | (96) | — |
| FJI-C11 | Ion-exchange | 93% | (3) | 155 | 3 min | (95) | — |

| | | | | | | |
|---|---|---|---|---|---|---|
| MOF-1-Cl | Ion-exchange | — | 31 | 30 min | (100) | — |

| | | | | | | |
|---|---|---|---|---|---|---|
| UiO-66-NH₂ @ Silica | Ion-exchange | 100% | (8) | 134 | — | 1 |

| | | | | | | |
|---|---|---|---|---|---|---|
| iMOF-2C | Ion-exchange | — | 214 | 25 min | (100) | — |

TABLE 8
Comparison of the binding mechanism, percentage of adsorption capacity
maintained upon adsorption-desorption regeneration cycles, saturation
adsorption capacity, and kinetic uptake rates for As(V) among PAF-
1-NMDG and reported state-of-the-art adsorbents. The pH condition
implemented for each reported value is also included.

| | | | | | Time to Reach | | |
|---|---|---|---|---|---|---|---|
| | | | | | Saturation | | |
| | | | | | (% of | | |
| | | | | As(V) | equilibrium | | |
| | Binding | Regenerability | | Capacity | capacity reached | | |
| Material | Mechanism | (Cycles) | | (mg/g) | at time) | | pH |
| PAF-1-NMDG | Ion exchange, | 100% | (10) | 94.2 | — | | 4 |
| | chelation | | | | | | |
| PAF-1-NMDG | Ion-exchange, | 100% | (10) | 85.5 | <10 s | (100) | 7 |
| | chelation | | | | | | |
| IRA743 Resin | Ion-exchange, | — | | 46.7 | 4 h | (100) | 7 |
| | chelation | | | | | | |
| Fe₂O₃ and MnO | Ion-exchange | 65.5% | (4) | 1.5 | 12 h | (97) | 7 |
| on Honeycomb | | | | | | | |
| Briquette Cinders | | | | | | | |
| MOF-808 | Ion-exchange | 82.1% | (5) | 24.8 | 50 min | (100) | 4 |
| UiO-66 | Ion-exchange | — | | 147.7 | 48 h | (100) | 7 |
| Fe-BTC | Fe | — | | 12.3 | 10 min | (100) | 4 |
| | complexation | | | | | | |
| MIL-53 (Al) | Ion-exchange | — | | 106.5 | 50 h | (100) | 8 |
| | hydrogen- | | | | | | |
| | bonding | | | | | | |
| Crosslinked | Polyol | — | | 16.4 | 24 h | (90) | 6 |
| NMDG and | complexation | | | | | | |
| Poly(vinylbenzene | | | | | | | |
| chloride) | | | | | | | |
| Fe-treated | Fe | 80% | (4) | 33.2 | 10 h | (100) | 7 |
| Cellulose | complexation | | | | | | |
| Fe-modified | Fe | — | | 51.3 | 24 h | (100) | 6 |
| Activated | complexation | | | | | | |
| Charcoal | | | | | | | |
| Fe(0) | Fe | 92% | (5) | 119 | 3 h | (100) | 7 |
| Nanoparticles on | complexation | | | | | | |
| Chitosan | | | | | | | |
| Molybdate | Mo | — | | 230 | 24 h | (100) | 2-3 |
| Chitosan | complexation | | | | | | |
| Derivatives | | | | | | | |
| H-MFI-90 | Ion-exchange | 97% | (1) | 34.8 | 100 min | (100) | 6.5 |

| Synthetic Zeolite | | | | | | |
|---|---|---|---|---|---|---|
| H-MFI-24 | Ion-exchange | — | 35.8 | 100 min | (100) | 6.5 |
| Synthetic Zeolite | | | | | | |
| Ultrafine Fe₂O₃ | Fe | — | 47 | 2 h | (95) | 7 |
| Nanoparticles | complexation | | | | | |
| Poly(glycidyl | Ion-exchange, | — | 45.9 | — | | 3 |
| methacrylate-N- | polyol | | | | | |
| methyl-D- | complexation | | | | | |
| glucamine) | | | | | | |

It will be understood that various modifications may be made without departing from the spirit and scope of this disclosure. Accordingly, other embodiments are within the scope of the following claims.

1. A method for the selective capture or separation of anionic compound(s), and/or anionic species, comprising:
contacting the anionic compound(s), and/or the anionic species with a porous adsorbent that has been functionalized to comprise pendant amino-diol groups and/or amino-polyol groups.

2. The method of claim 1, wherein the porous adsorbent is selected from porous an organic polymer, a porous metal particle, a porous metal oxide particle, a metal organic framework (MOF), a zeolitic organic framework (ZIF), a covalent organic framework (COF), and a porous aromatic framework (PAF).

3. (canceled)

4. The method of claim 2, wherein the PAF have tetrahedral carbon nodes connected by a plurality of linkers having the structure of: 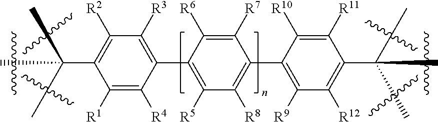
wherein,
R¹-R¹² are each independently selected from H, D, an optionally substituted functional group (FG), an optionally substituted (C₁-C₉)alkyl, an optionally substituted (C₁-C₉)alkenyl, an optionally substituted (C₁-C₉) alkynyl, an optionally substituted (C₁-C₈) heteroalkyl, an optionally substituted (C₁-C₅) heteroalkenyl, an optionally substituted (C₁-C₈) heteroalkynyl, an optionally substituted aryl, an optionally substituted heterocycle, wherein at least one of R¹-R¹² is an optionally substituted FG that comprises an amino-diol group or an amino-polyol group; and
n is an integer selected from 0, 1, or 2.

5. The method of claim 4, wherein the amino-diol group or the amino-polyol group has a structure selected from:

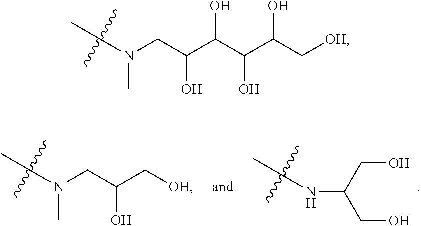

6. The method of claim 2, wherein the PAF has tetrahedral carbon nodes connected by a plurality of linkers having the structure of: 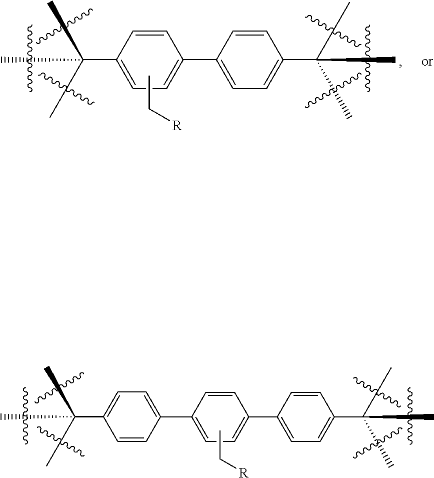
wherein,
R is selected from

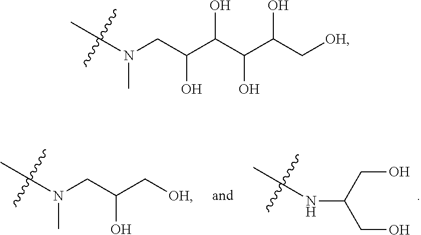

7. The method of claim 6, wherein R is

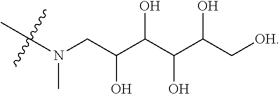 [SMILES: *N(C)CC(O)C(O)C(O)C(O)CO]

8. The method of claim 1, wherein the adsorbent can be used to selectively capture or separate anionic compounds, and/or an anionic species comprising Cr(0), Cr(II), Cr(III), Cr(IV), Cr(VI), As(0), As(III), and/or As(V).

9. The method of claim 8, wherein the anionic compounds, and/or an anionic species are selected from CrO₄²⁻, HCrO₄⁻, H₂CrO₄, Cr₂O₇²⁻, HCr₂O₇⁻, and/or H₂Cr₂O₇.

10. (canceled)

11. The method of claim 8, wherein the anionic compounds, and/or an anionic species are selected from AsO₄³⁻, HAsO₄²⁻, H₂AsO⁴⁻, H₃AsO₄, H₃AsO₃, H₂AsO³⁻, HAsO₃²⁻, and/or AsO₃³⁻.

12. The method of claim 1, wherein the anionic compound(s), and/or anionic species are selectively captured or separated from a fluid stream.

13. The method of claim 12, wherein the fluid stream is gas mixture or a water source.

14. (canceled)

15. The method of claim 1, wherein the anionic compound(s), and/or anionic species are waterborne oxyanions.

16. The method of claim 15, wherein the waterborne oxyanions are selected from vanadate, molybdate, permanganate, tungstate, antimony oxyanions, ferrate, rhenate, perrhenate, tellurate, tellurite, selenate, selenite, perxenate, carbonate, bicarbonate, phosphite, phosphate, hyposulfite, nitrite, nitrate, chlorate, sulfate, thiosulfate, acetate, perchlorate, hypochlorite, iodate, bromite, perbromate, bromate, and hypobromite.

17. A device comprising a porous adsorbent that has been functionalized to comprise pendant amino-diol groups and/or amino-polyol groups.

18. The device of claim 17, wherein the porous adsorbent is selected from porous an organic polymer, a porous metal particle, a porous metal oxide particle, a metal organic framework (MOF), a zeolitic organic framework (ZIF), a covalent organic framework (MOF), and a porous aromatic framework (PAF).

19. (canceled)

20. The device of claim 18, wherein the PAF has tetrahedral carbon nodes connected by a plurality of linkers having the structure of: 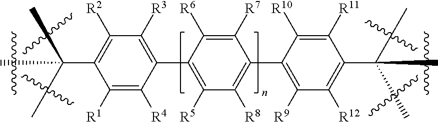
wherein,
R¹-R¹² are each independently selected from H, D, an optionally substituted functional group (FG), an optionally substituted (C₁-C₉)alkyl, an optionally substituted (C₁-C₉)alkenyl, an optionally substituted (C₁-C₉) alkynyl, an optionally substituted (C₁-C₅) heteroalkyl, an optionally substituted (C₁-C₅) heteroalkenyl, an optionally substituted (C₁-C₈) heteroalkynyl, an optionally substituted aryl, an optionally substituted heterocycle, wherein at least one of R¹-R¹² is an optionally substituted FG that comprises an amino-diol group or an amino-polyol group; and
n is an integer selected from 0, 1, or 2.

21. The device of claim 20, wherein the amino-diol group or the amino-polyol group has a structure selected from:

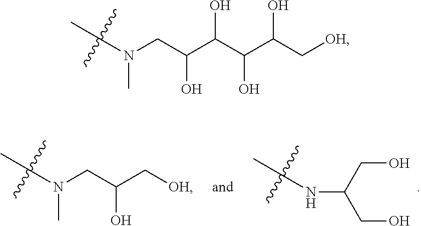

22. The device of claim 18, wherein the PAF has tetrahedral carbon nodes connected by a plurality of linkers having the structure of: 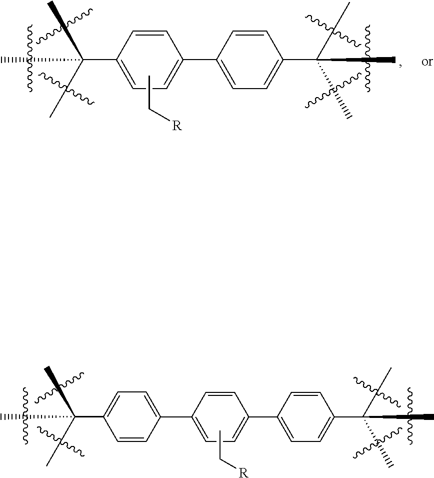
wherein,
R is selected from

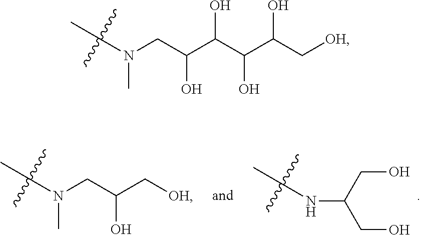

23. The device of claim 22, wherein R is

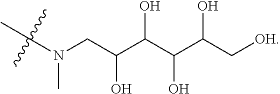 [SMILES: *N(C)CC(O)C(O)C(O)C(O)CO]

24. The device of claim 17, wherein the adsorbent can be used to selectively capture or separate anionic compounds, and/or an anionic species comprising Cr(0), Cr(II), Cr(III), Cr(IV), Cr(VI), As(0), As(III), and/or As(V).

25. The device of claim 24, wherein the anionic compounds, and/or an anionic species are selected from CrO₄²⁻, HCrO₄⁻, H₂CrO₄, Cr₂O₇²⁻, HCr₂O₇⁻, and/or H₂Cr₂O₇.

26. (canceled)

27. The device of claim 24, wherein the anionic compounds, and/or an anionic species are selected from AsO₄³⁻, HAsO₄²⁻, H₂AsO⁴⁻, H₃AsO₄, H₃AsO₃, H₂AsO³⁻, HAsO₃²⁻, and/or AsO₃³⁻.

28. The device of claim 17, wherein the device is a storage device that stores the captured anionic compounds and/or anionic species.

29. The device of claim 17, wherein the device is a separation or a sensor device that selectively separates, or indicates the capture, of anionic compounds and/or anionic species.

30. The device of claim 29, wherein the adsorbent is integrated into membranes, films, electrodes, coatings, pellets, co-polymers, porous substrates, or indicators.

31. A functionalized porous organic framework (PAF) having tetrahedral carbon nodes connected by linkers having the general structure of: 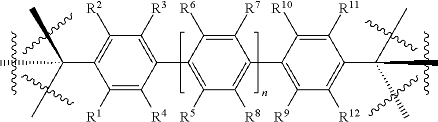
wherein,
R¹-R¹² are each independently selected from H, D, an optionally substituted functional group (FG), an optionally substituted (C₁-C₉)alkyl, an optionally substituted (C₁-C₉)alkenyl, an optionally substituted (C₁-C₉)alkynyl, an optionally substituted (C₁-C₅) heteroalkyl, an optionally substituted (C₁-C₅) heteroalkenyl, an optionally substituted (C₁-C₅) heteroalkynyl, an optionally substituted cycloalkyl, an optionally substituted aryl, an optionally substituted heterocycle, wherein at least one of R¹-R¹² is an FG comprising a secondary or tertiary amine; and
n is an integer selected from 0, 1, 2, or 3;
wherein, the optionally substituted FG comprising a secondary and/or a tertiary amine does not have the structure of

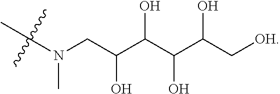 [SMILES: *N(C)CC(O)C(O)C(O)C(O)CO]

32. The functionalized PAF of claim 31, wherein at least one of R¹-R¹² is an optionally substituted FG having the structure of 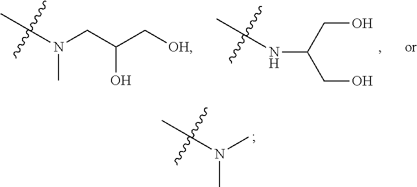
and
n is an integer selected from 0, 1, 2, or 3.

33. The functionalized PAF of claim 31, wherein linkers have the structure of: 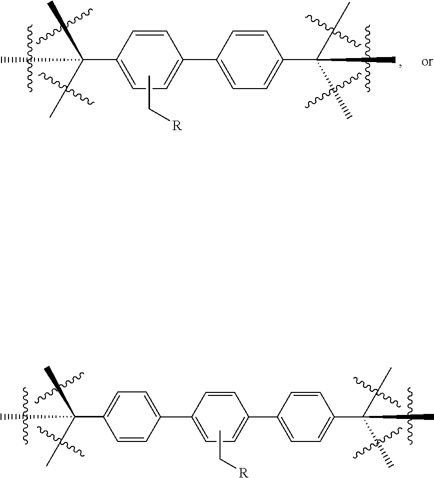
wherein,
R is selected from [chemical structure]
